(19) United States
(12) Patent Application Publication
(10) Pub. No.: US 2003/0032074 A1
(43) Pub. Date: Feb. 13, 2003

(54) Methods to determine prognosis after therapy for bladder cancer

(76) Inventors: Kevin M. Slawin, Houston, TX (US); Shahrokh Shariat, Dallas, TX (US); Seth P. Lerner, Houston, TX (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/156,667

(22) Filed: May 24, 2002

Related U.S. Application Data
(60) Provisional application No. 60/295,512, filed on Jun. 1, 2001.

(51) Int. Cl. G01N 33/574

(52) U.S. Cl. 435/7.23

(57) ABSTRACT

A method to diagnose, stage and predict prognosis of bladder cancer patients is provided, e.g., using TGF-β1, IL-6, IL-6sR, uPA, uPAR and IGFBP-3. These markers, and potentially others, in combination with standard clinical and pathologic features, may be used to create a nomogram that would be useful for managing bladder cancer patients.

### CROSS-REFERENCE TO RELATED APPLICATIONS

This application claims the benefit of the filing date of U.S. application Serial No. 60/295,512, filed Jun. 1, 2001 under 35 U.S.C. §119(e).

### STATEMENT OF GOVERNMENT RIGHTS

The invention was made at least in part with a grant from the Government of the United States of America (grant no. CA 58203 from the National Institutes of Health). The Government has certain rights to the invention.

### BACKGROUND OF THE INVENTION

Carcinoma of the urinary bladder is a significant cause of morbidity and mortality in the United States, accounting for an estimated 54,300 new cases and 12,400 resultant deaths in 2001 (Greenlee et al., 2001). At initial presentation, approximately 70% to 80% of the bladder cancers are confined to the epithelium or subepithelial connective tissue, whereas the remainder of patients present with muscle invasive cancer. Most of these patients respond well to local resection and adjuvant intravesical therapy. Unfortunately, 50% to 80% of these patients will experience recurrence within the first two years. Up to one-half of patients with invasive disease have either measurable metastatic disease or have disease destined to recur due to occult metastases. While patients with low-grade papillary disease (Ta, T1) rarely progress to muscle invasive transitional cell carcinoma (TCC), as many as 30% of patients with Tis or high-grade papillary disease are refractory to treatment with intravesical immunotherapy and/or chemotherapy and progress to more advance disease (Thrasher and Crawford, 1993).

Radical cystectomy provides effective local control of refractory Ta, T1 and Tis bladder cancer and muscle invasive TCC (Stein et al., 2001). However, despite this, approximately 14%-33% of patients with node-negative muscle invasive disease will die of their disease within five years of surgery, presumably due to dissemination of microscopic metastatic disease prior to cystectomy (Lerner and Skinner, 1999). Patients who have pathologically confirmed metastases to regional lymph nodes have an even higher risk of progression and death than those who do not (Lerner et al., 1993). Ultimately at least 50% of the patients undergoing cystectomy for muscle invasive disease will eventually develop clinical metastases and most of these patients will die of their disease (Raghavan et al., 1990; von der Maase et al., 2000). Conventional staging modalities such as transurethral bladder tumor resection (Lerner et al., 1993; Frazier et al, 1993; Pagano et al., 1991), computed tomography (Buszello et al., 1994; Nurmi et al. 1988, Voges et al., 1989), and magnetic resonance imaging (Persad et al., 1993) have a limited role in tumor staging and predicting lymph node involvement in patients with bladder cancer because of their poor performance in detecting early, small-volume metastases. Early detection of patients harboring occult micrometasteses, who have a high probability of developing disease progression and shortened survival, could potentially be spared either the morbidity of an unsuccessful local treatment or selected for a combined modality program.

Cystoscopy is the mainstay of surveillance for Ta, Tis and T1 bladder malignancies and is usually supplemented with urine cytology. Voided urine cytology has excellent specificity with few false positive results (Brown, 2000). Its sensitivity is about 70% but decreases to 30% to 50% for low-grade tumors (Murphy, et al., 1984). Recently, a variety of urinary markers purported to be more sensitive than urine cytology have been investigated. A common problem of these markers is that when they have a reasonable to good sensitivity, the specificity remains relatively low. Consequently, cytology is still considered the gold standard for non-muscle-invasive bladder malignancy screening (Brown, 2000).

Serum markers that have been associated with a variety of malignancies or with an increased risk for certain cancers include transforming growth factor beta (TGF-β), interleukin 6 (IL-6), insulin-like growth factor (IGF) and urokinase-type plasminogen activator (uPA).

TGF-β₁ is a pleiotropic growth factor that regulates cellular proliferation, chemotaxis, cellular differentiation, immune response, and angiogenesis. Loss of response to the antiproliferative effects of TGF-β₁ has been associated with the later stages of carcinogenic progression. Increased local expression of TGF-β₁ has been associated with pathologic features of aggressive bladder cancer such as high tumor grade, advanced stage, and lymphovascular invasion, as well as with disease progression (Kim et al., 2001). Elevated circulating levels of TGF-β₁ have been found in patients with a variety of malignancies (Shirai et al., 1994; Junker et al., 1996), and have been associated with invasion, progression, and metastases (Kong et al., 1999; Adler et al., 1999; Ivanovic et al., 1995; Kakehi et al., 1996; Shariat et al., 2001c). Similarly, TGF-β₁ levels have been found to be elevated in patients with invasive bladder cancer (Eder et al., 1996).

IL-6 is a pleiotropic cytokine involved in mediating cell proliferation and host response to tissue injury. The major activities of IL-6 include the proliferation and activation of cytotoxic T cells, proliferation and differentiation of B cells, and stimulation of acute-phase proteins (Sehgal, 1990). IL-6 signals by binding with a ligand-specific receptor (IL-6R), triggering the homodimerization of a transmembrane signal-transducing receptor (gp130). This phosphorylates associated tyrosine kinases (JAKI, JAK2, and TYK2) to initiate the JAK-STAT pathway. The soluble form of IL-6R (IL-6sR) arises from two independent cellular processes: differential mRNA splicing and proteolytic cleavage of membrane-bound IL-6 receptors. IL-6sR can augment IL-6-induced signaling by a transsignaling process in which the IL6/IL-6sR complex binds to membrane-bound gp130 (Jones et al., 2001).

Increased local IL-6 expression has been demonstrated in cancer cell lines and tumors from various cancers including renal (Takenawa et al., 1991), prostate (Chung et al., 1999), colorectal (Kinoshita et al., 1999), and bladder (Okamoto et al., 1997). Furthermore, elevated circulating levels of IL-6 have been found in metastatic cancer patients (Shariat et al., 2001a; Adler et al., 1999; Akimoto et al., 1998; Ueda et al., 1994) and have been associated with increased mortality due to prostate (Nakashima et al., 2000), colorectal (Belluco et al., 2000), and ovarian cancers (Scambia et al., 1995).

IGFs are mitogens that have been shown to play a significant role in regulating proliferation and differentiation of bladder cancer cells. Recently, a number of epidemiological studies have shown that higher circulating levels of IGF-I and lower circulating levels of IGF binding protein-3 (IGFBP-3) are associated with an increased risk for the development of several common cancers.

uPA is a serine protease that is involved in the formation and maintenance of blood vessels and clots, bone remodeling and the activation of metalloproteinases and growth factors. uPA may also play a role in tumor invasion and metastases by initially catalyzing the conversion of plasminogen to plasmin (Wang et al., 2001; Andreason et al., 2000). Plasmin cleaves components of the basement membrane and extracellular matrix, such as collagen, fibronectin, and laminin, thereby allowing tumor cells access to lymph vessels and vasculature. uPA can also directly degrade fibronectin in the extracellular matrix and activate collagenase IV. The inactive precursor of uPA is activated by binding to a specific membrane bound or soluble cell surface receptor, uPAR, which accelerates the conversion of plaminogen into plasmin (Wang et al., 2001; Andreason et al., 2000). Hudson et al. (1997) demonstrated that the presence of both uPA and uPAR is necessary for bladder tumor cell invasion in vitro. Elevated local and circulating levels of uPA and uPAR, have been associated with poor clinical outcome in several cancers (Duffy et al., 1999). In bladder cancer, uPA is produced locally by cancer cells, and increased local levels of uPA have been associated with decrease survival in patients with Ta and T1 TCC (Hudson et al., 1997; Hasui et al., 1996).

Unlike prostate cancer, where prostate specific antigen (PSA) has become an indispensable tool for disease management because of a significant association between PSA levels and cancer stage, risk of disease progression, and response to therapy, bladder cancer lacks a clinically useful biomarker for predicting disease stage and clinical outcome. While cell surface markers such as CEA, CA-125 and βHCG are frequently elevated in the serum of patients with locally advanced and metastatic bladder cancer (Izes et al., 2001), and studies involving circulating levels of tumor-related proteins such as matrix metalloproteinase-2 (Gohji et al., 1996), hepatocyte growth factor (Gohji et al., 2000), and tissue polypeptide antigen (Maulard-Durdux et al., 1997) have shown promise, none of these markers has been shown to have clinical applications. That is, none of these markers has been shown, when quantitated prior to therapy, to aid in treatment planning and risk stratification, or when quantitated during therapy, to aid in assessing treatment response.

### SUMMARY OF THE INVENTION

The invention provides a method to predict disease outcome for patients with bladder cancer. In one embodiment of the invention, the method comprises contacting a physiological fluid sample from a patient prior to therapy, e.g., prior to radical cystectomy, for bladder cancer with an agent that binds to TGF-β₁ so as to form a complex. The amount or level of complex formation is then correlated to the risk of metastases, regional or distant, to the lymph node, lymphovascular invasion, disease recurrence or disease-specific mortality in the patient. As used herein, a “physiological fluid sample” includes, but is not limited to, a sample of blood, blood plasma, blood serum, seminal fluid, urine, saliva, sputum, semen, pleural effusions, bladder washes, bronchioalveolar ravages, cerebrospinal fluid and the like. In one embodiment of the invention, a blood sample, preferably a plasma sample, is contacted with an agent that binds TGF-β₁. Preferred agents that bind to TGF-β₁ include, but are not limited to, antibodies specific for TGF-β₁ and the TGF-β₁ receptor protein, e.g., type I or II, although other agents that bind TGF-β₁, may be employed.

As described herein, pre-operative TGF-β₁ levels were measured in 51 patients who underwent radical cystectomy (median follow-up of 45.7 months) for muscle invasive or intravesical immunotherapy and/or chemotherapy refractory Tis, Ta or T1 TCC, and correlated with pathological parameters and clinical outcome. TGF-β₁ levels were also measured in 44 healthy men without any cancer. Plasma TGF-β₁ levels in patients who eventually developed metastases to distant (11.9±0.9 ng/mL) or regional (9.6±2.4 ng/mL) lymph nodes were significantly higher than that in patients with non-metastatic muscle invasive TCC (5.4±1.1 ng/mL), which in turn was significantly higher than that in patients with non-metastatic Tis, Ta or T1 TCC (4.5±1.2 ng/mL) and healthy subjects (4.5±1.2 ng/mL) (P values<0.001). Pre-operative plasma TGF-β₁ level was an independent predictor of lymphovascular invasion (P=0.002), metastases to lymph nodes (P=0.030), disease recurrence (P=0.009), and disease specific survival (P=0.015). In a subgroup of patients with muscle invasive TCC, TGF-β₁ level was associated with disease recurrence (P=0.005) and death from bladder cancer (P=0.001). TGF-β₁ levels were highest in patients with bladder cancer metastatic to lymph nodes and are a strong independent predictor of disease recurrence and disease-specific mortality. Thus, the level of TGF-β₁ in body fluids of humans is prognostically useful, and may optionally be employed in conjunction with other markers (clinicopathological measurements) of neoplastic disease, e.g., in a nomogram to predict stage and outcome in patients with bladder cancer. In one embodiment, the prognosis is based on a computer-derived analysis of data of the amount, level or other value (score) for one or more markers for bladder cancer. Data may be input manually or obtained automatically from an apparatus for measuring the amount or level of one or more markers.

As also described herein, the pre-operative plasma levels of IL-6 and IL-6sR were measured in the 51 patients who underwent radical cystectomy for TCC and in the 44 healthy men. IL-6 levels were higher in bladder cancer patients than in healthy controls (P<0.001). In bladder cancer patients, elevated levels of both IL-6 and IL-6sR were associated with adverse pathological features including muscle invasive disease, lymphovascular invasion, and lymph node metastases (P values<0.05). High levels of IL-6sR were also associated with pathological tumor grade (P=0.036). In separate multivariate models that included clinical stage and grade, IL-6 and IL-6sR levels were independent predictors of lymphovascular invasion, metastases to lymph nodes, disease recurrence, and disease-specific survival (P values<0.05). In a preoperative Cox proportional hazards model, while both IL-6 (P=0.050) and IL-6sR (P=0.035) predicted disease-specific survival, only IL-6sR predicted disease recurrence (P=0.042). Thus, levels of IL-6 and IL-6sR were associated with cancer stage and metastases and were strong independent predictors of disease recurrence and disease-specific mortality. As the level of IL-6 and/or IL-6sR in body fluids of humans is prognostically useful, the invention further provides a method in which a physiological fluid sample from a patient prior to radical cystectomy for bladder cancer is contacted with an agent that binds to IL-6 or IL-6sR so as to form a complex. Then the amount or level of complex formation is correlated to disease outcome. The level of IL-6 and/or IL-6sR in physiologic fluids may optionally be employed in conjunction with other markers for neoplastic disease, e.g., in a nomogram to predict grade and outcome in patients with bladder cancer. In one embodiment, the prognosis may be based on a computer derived analysis of data of the amount, level or other value for one or more markers for bladder cancer, and data may be input manually or obtained automatically.

As further described herein, pre-operative plasma levels of IGF-I and IGFBP-3 were measured in 51 patients who underwent radical cystectomy for bladder cancer and in 44 healthy men. Plasma IGF-I and IGFBP-3 levels in bladder cancer patients were not significantly different than those in healthy subjects (P values≧0.339). In a pre-operative model that included clinical grade, clinical stage, and either IGF-I or IGFBP-3, none of the parameters was associated with either metastases to lymph nodes or clinical outcome. However, in an alternative model that included both IGF-I and IGFBP-3, pre-operative plasma IGFBP-3 was an independent predictor of metastases to regional lymph nodes, bladder cancer progression and survival (P=0.047, P=0.050, and P=0.040, respectfully). Hence, pre-operative plasma IGFBP-3 level is a predictor of metastases to lymph nodes and clinical outcome after radical cystectomy when adjusted for IGF-I level.

The invention thus provides a method which comprises contacting a physiological fluid sample from a patient prior to radical cystectomy for bladder cancer with an agent that binds to IGFBP-3, so as to form a complex. Complex formation is then detected or determined, and correlated to disease outcome. Similar to the methods described above, the level of IGFBP-3 in body fluids of humans is prognostically useful, and may optionally be employed in conjunction with other markers for bladder cancer to predict outcome in patients with bladder cancer, e.g., using a computer derived analysis of data of the amount, level or other value for one or more markers for bladder cancer.

As further described herein, pre-operative plasma levels of uPA and uPAR were measured in 51 patients who underwent radical cystectomy for muscle invasive cancer or Tis, Ta or T1 TCC refractory to intravesical therapy. Plasma uPA and uPAR levels were both higher in TCC patients compared to healthy subjects (P<0.001). Pre-operative plasma uPAR levels were highest in patients with distant metastases to distant lymph nodes (P=0.042). Pre-operative plasma levels of uPA were independently associated with the presence of metastases in regional lymph nodes (P=0.017), lymphovascular invasion (P=0.019), disease progression (P=0.030) and death from bladder cancer (P=0.038). 19/51(37%) patients recurred and 12/51 (23%) patients died from bladder cancer. Plasma uPAR was not associated with bladder cancer outcome. Thus, uPA in physiologic fluids such as plasma is an independent predictor of clinical outcome in patients with TCC.

An enzyme-linked immunosorbent assay was used to compare voided urine levels of uPA and uPAR collected before cystoscopy in 122 bladder cancer patients and 107 controls. Seventy two (72) patients had clinical Tis or Ta TCC and 50 had invasive disease (≧T1); 85 patients had clinical grade 1 or 2 tumors and 37 had grade 3 tumors. For cytology, only high grade was considered positive. Urinary levels of uPA and uPAR were higher in cancer patients than in controls (P<0.001 and P=0.016, respectively). However, only uPA levels were elevated in patients with abnormal urinary cytology (P=0.006). After controlling for cytology (OR 10.182, 95% CI 4.451-23.291, P<0.001), uPAR (P for trend=0.168), and age (P=0.091), subjects in the highest quartile for uPA had an increased risk of bladder cancer compared with subjects in the lowest quartile (OR 3.022, 95%CI 1.295-7.054, P for trend=0.031).

The invention thus provides a method which comprises contacting a physiological fluid sample from a patient prior to radical cystectomy for bladder cancer with an agent that binds to uPA or uPAR, so as to form a complex. Complex formation is then detected or determined, and correlated to disease outcome. Similar to the methods described above, the level of uPA or uPAR in body fluids of humans is prognostically useful, and may optionally be employed in conjunction with other markers for bladder to predict outcome in patients with bladder cancer, e.g., using a computer derived analysis of data of the amount, level or other value for one or more markers for bladder cancer.

The invention also provides an apparatus, comprising: a data input means, for input of test information comprising the level or amount of at least one protein in a sample obtained from a mammal, wherein the protein is selected from the group consisting of TGF-β₁, IGF-I, IGFBP-3, IL-6, IL-6sR, uPA and/or uPAR; a processor, executing a software for analysis of the level or amount of the at least one protein in the sample; wherein the software analyzes the level or amount of the at least one protein in the sample and provides the risk of metastases to the lymph node, disease recurrence or disease-specific mortality in the mammal.

Another embodiment of the invention is a method to diagnose bladder cancer in a patient. The method comprises contacting a physiological fluid sample, e.g., a blood plasma sample, from a patient with an agent that binds to IL-6 so as to form a complex. The amount or level of complex formation is correlated with the presence or absence of bladder cancer.

Another embodiment of the invention is a method to diagnose bladder cancer in a patient in which a physiological fluid sample, e.g., a urine sample, from a patient in contacted with an agent that binds to uPA so as to form a complex. The amount or level of complex formation is correlated with the presence or absence of bladder cancer.

### BRIEF DESCRIPTION OF THE FIGURES

FIG. 1A. Kaplan-Meier estimates of the probability of disease recurrence following radical cystectomy according to the median TGF-β₁ level of 5.0 ng/mL.

FIG. 1B. Kaplan-Meier estimates of the probability of bladder cancer-specific survival following radical cystectomy according to the median TGF-β₁ of 5.0 ng/mL.

FIG. 2A. Graph of ranges of plasma TGF-β₁ levels in healthy men; Ta, T1 and CIS patients; muscle invasive TCC patients; bladder cancer patients with lymph node metastases; and bladder cancer patients with distant lymph node metastases.

FIG. 2B. Pre-operative plasma TGF-β₁ level correlation with clinical and pathologic characteristics.

FIG. 3A. Survival analysis according to the median TGF-β₁ (DMOS=follow-up time since surgery).

FIG. 3B. Mulivariate analyses of pre-operative features including TGF-β₁ levels for the prediction of disease recurrence and disease-specific survival.

FIG. 3C. Multivariate analyses of post-operative features including TGF-β₁ levels for the prediction of disease recurrence and disease-specific survival.

FIGS. 4A-B. Kaplan-Meier estimates of the probability of disease recurrence (A) and bladder cancer-specific mortality (B) following radical cystectomy according to the median IL-6 level of 4.80 pg/mL.

FIGS. 5A-B. Kaplan-Meier estimates of the probability of disease recurrence (A) and bladder cancer-specific mortality (B) following radical cystectomy according to the median IL-6sR level of 21.23 ng/mL.

FIGS. 6A-B. Kaplan-Meier estimates of the probability of disease recurrence (A) and bladder cancer-specific mortality (B) following radical cystectomy according to the median IL-6 level of 4.80 pg/mL and median IL-6sR level of 21.23 ng/mL.

FIG. 7. Survival analysis according to the median IL-6sR (DMOS follow-up time since surgery).

FIG. 8. Comparison of IGF-I and IGFBP-3 levels in patients undergoing radical cystectomy versus control patients.

FIG. 9. Kaplan-Meier estimates of the probability of disease progression following radical cystectomy according to the median IGF-I level of 167 ng/mL.

FIG. 10. Kaplan-Meier estimates of the probability of disease progression following radical cystectomy according to the median IGFBP-3 level of 3052 ng/mL.

FIG. 11. Kaplan-Meier estimates of the probability of bladder cancer-specific survival following radical cystectomy according to the median IGF-I level of 167 ng/mL.

FIG. 12. Kaplan-Meier estimates of the probability of bladder cancer-specific survival following radical cystectomy according to the median IGFBP-3 level of 3052 ng/mL.

FIG. 13. Multivariate analyses of clinical prognostic factors for recurrence and death from bladder cancer.

### DETAILED DESCRIPTION OF THE INVENTION

The invention in its broadest sense is a method for predicting organ confined (local) disease status or the potential for progression of bladder cancer following primary therapy (cystectomy), e.g., the presence of metastases, disease recurrence or disease-specific mortality. The method is particularly useful for evaluating patients at risk for recurrence of bladder cancer following primary therapy for bladder cancer. Specifically, the detection of pre-operative TGF-β₁, IL-6, IL-6sR, IGF-I, IGFBP-3, uPA or uPAR levels alone, or in conjunction with other markers for bladder cancer, are useful in predicting metastases to the lymph nodes, disease recurrence and/or disease-specific mortality.

Non-invasive prognostic assays are provided by the invention to detect and/or quantitate TGF-β₁, IL-6, IL-6sR, IGF-I, IGFBP-3, uPA and/or uPAR levels in the body fluids of mammals, including humans. Thus, such an assay is useful in prognosis of bladder cancer. Moreover, such assays provide valuable means of monitoring the status of the bladder cancer. In addition to improving prognostication, knowledge of the disease status allows the attending physician to select the most appropriate therapy for the individual patient. For example, patients with a high likelihood of relapse can be treated rigorously. Because of the severe patient distress caused by the more aggressive therapy regimens as well as cystectomy, it would be desirable to distinguish with a high degree of certainty those patients requiring aggressive therapies as well as those which will benefit from cystectomy.

The body fluids that are of particular interest as physiological samples in assaying for TGF-β₁, IL-6, IL-6sR, IGF-I, IGFBP-3, uPA and/or uPAR according to the methods of this invention include blood, blood serum, semen, saliva, sputum, urine, blood plasma, pleural effusions, bladder washes, bronchioalveolar lavages, and cerebrospinal fluid. Urine, blood, serum and plasma are preferred, and plasma, such as platelet-poor plasma, are the more preferred samples for use in the methods of this invention.

Exemplary means for detecting and/or quantitating TGF-β₁, IL-6, IL-6sR, IGF-I, IGFBP-3, uPA and/or uPAR levels in mammalian body fluids include electrophoresis, e.g., capillary electrophoresis, affinity chromatography, Western blot analysis, immunoprecipitation analysis, and immunoassays, including ELISAs (enzyme-linked immunosorbent assays), RIA (radioimmunoassay), competitive EIA or dual antibody sandwich assays. In such immunoassays, the interpretation of the results is based on the assumption that the TGF-β₁, IL-6, IL-6sR, IGF-I, IGFBP-3, uPA or uPAR binding agent, e.g., a TGF-β₁-, IL-6-, IL-6sR-, IGF-1-, IGFBP-3-, uPA- or uPAR-specific antibody, will not cross-react with other proteins and protein fragments present in the sample that are unrelated to TGF-β₁, IL-6, IL-6sR, IGF-I, IGFBP-3, uPA or uPAR. Preferably, the method used to detect TGF-β₁, IL-6, IL-6sR, IGF-I, IGFBP-3, uPA and/or uPAR levels employs at least one TGF-β₁, IL-6-, IL-6sR-, IGF-1-, IGFBP-3-, uPA- or uPAR-specific binding molecule, e.g., an antibody or at least a portion of the ligand for any of those molecules. Immunoassays are a preferred means to detect TGF-β₁, IL-6, IL-6sR, IGF-I, IGFBP-3, uPA and/or uPAR. Representative immunoassays involve the use of at least one monoclonal or polyclonal antibody to detect and/or quantitate TGF-β₁, IL-6, IL-6sR, IGF-I, IGFBP-3, uPA or uPAR in the body fluids of mammals. The antibodies or other binding molecules employed in the assays may be labeled or unlabeled. Unlabeled antibodies may be employed in agglutination; labeled antibodies or other binding molecules may be employed in a wide variety of assays, employing a wide variety of labels.

Suitable detection means include the use of labels such as radionuclides, enzymes, fluorescers, chemiluminescers, bioluminescers, enzyme substrates or co-factors, enzyme inhibitors, particles, dyes and the like. Such labeled reagents may be used in a variety of well known assays. See for example, U.S. Pat. Nos. 3,766,162, 3,791,932, 3,817,837, and 4,233,402.

Still further, in, for example, a competitive assay format, labeled TGF-β₁, IL-6, IL-6sR, IGF-I, IGFBP-3, uPA or uPAR peptides and/or polypeptides can be used to detect and/or quantitate TGF-β₁, IL-6, IL-6sR, IGF-I, IGFBP-3, uPA or uPAR, respectively, in mammalian body fluids. Also, alternatively, as a replacement for the labeled peptides and/or polypeptides in such a representative competitive assay, labeled anti-idiotype antibodies that have been prepared against antibodies reactive with TGF-β₁, IL-6, IL-6sR, IGF-I, IGFBP-3, uPA or uPAR can be used.

It can be appreciated that certain molecules such as TGF-β₁ may be present in various forms, e.g., latent and active, as well as fragments thereof, and that these various forms may be detected and/or quantitated by the methods of the invention if they contain one or more epitopes recognized by the respective binding agents. For example, in a sandwich assay where two antibodies are used as a capture and a detection antibody, respectively, if both epitopes recognized by those antibodies are present on at least one form of, for example, TGF-β₁, the form would be detected and/or quantitated according to such an immunoassay. Such forms which are detected and/or quantitated according to methods of this invention are indicative of the presence of the active form in the sample.

For example, TGF-β₁, IL-6, IL-6sR, IGF-I, IGFBP-3, uPA or uPAR levels may be detected by an immunoassay such as a “sandwich” enzyme-linked immunoassay (see Dasch et al., 1990; Danielpour et al., 1989; Danielpour et al., 1990; Lucas et al., 1990; Thompson et al., 1989; and Flanders et al., 1989). A physiological fluid sample is contacted with at least one antibody specific for TGF-β₁, IL-6, IL-6sR, IGF-1, IGFBP-3, uPA or uPAR to form a complex with said antibody and TGF-β₁, IL-6, IL-6sR, IGF-I, IGFBP-3, uPA or uPAR. Then the amount of TGF-β₁, IL-6, IL-6sR, IGF-I, IGFBP-3, uPA or uPAR in the sample is measured by measuring the amount of complex formation. Representative of one type of ELISA test is a format wherein a solid surface, e.g., a microtiter plate, is coated with antibodies to TGF-β₁, IL-6, IL-6sR, IGF-I, IGFBP-3, uPA or uPAR and a physiological fluid sample of a patient is added to a well on the microtiter plate. After a period of incubation permitting any antigen to bind to the antibodies, the plate is washed and another set of TGF-β₁, IL-6, IL-6sR, IGF-I, IGFBP-3, uPA or uPAR antibodies, e.g., antibodies that are linked to a detectable molecule such as an enzyme, is added, incubated to allow a reaction to take place, and the plate is then rewashed. Thereafter, enzyme substrate is added to the microtiter plate and incubated for a period of time to allow the enzyme to catalyze the synthesis of a detectable product, and the product, e.g., the absorbance of the product, is measured.

It is also apparent to one skilled in the art that a combination of antibodies to TGF-β₁, IL-6, IL-6sR, IGF-I, IGFBP-3, uPA or uPAR can be used to detect and/or quantitate the presence of TGF-β₁, IL-6, IL-6sR, IGF-I, IGFBP-3, uPA or uPAR in the body fluids of patients. In one such embodiment, a competition immunoassay is used, wherein TGF-β₁, IL-6, IL-6sR, IGF-I, IGFBP-3, uPA or uPAR is labeled, and a body fluid is added to compete the binding of the labeled TGF-β₁, IL-6, IL-6sR, IGF-I, IGFBP-3, uPA or uPAR to antibodies specific for TGF-β₁, IL-6, IL-6sR, IGF-I, IGFBP-3, uPA or uPAR. Such an assay could be used to detect and/or quantitate TGF-β₁, IL-6, IL-6sR, IGF-I, IGFBP-3, uPA or uPAR.

Thus, once binding agents having suitable specificity have been prepared or are otherwise available, a wide variety of assay methods are available for determining the formation of specific complexes. Numerous competitive and non-competitive protein binding assays have been described in the scientific and patent literature and a large number of such assays are commercially available. Exemplary immunoassays which are suitable for detecting a serum antigen include those described in U.S. Pat. Nos. 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Methods to detect TGF-β₁ levels as well as TGF-β binding molecules are well known to the art (see, e.g., U.S. Pat. Nos. 5,216,126, 5,229,495, 5,571,714, and 5,578,703; WO 91/08291; WO 93/09228; WO 93/09800; and WO 96/36349).

The methods of the invention may be employed with other measures of bladder cancer biology to better predict disease-free status or for staging. The term “stage” is defined as the estimation of the extent (size and location) of the cancer. More specifically, how extensive is the cancer within the bladder and if it has spread to tissues around the bladder, or to other parts of the body. Clinical stage is defined as the stage estimated by the physician before any surgery is done, while pathologic stage is defined as the actual extent of the cancer as found by the pathologist in the bladder specimen after removal of the bladder and lymph nodes (if that option is performed). The following clinical and pathological staging criteria may be used, e.g., clinical and pathological stage, although the use of other criteria does not depart from the scope and spirit of the invention.

TX—Primary tumor cannot be assessed.

TO—No evidence of primary tumor.

Ta—Non-invasive papillary carcinoma.

Tis—Carcinoma in situ (“flat tumor”).

T1—Tumor invades subepithelial connective tissue.

T2—Tumor invades muscle.

T2a—Superficial muscle affected (inner half).

T2b —Deep muscle affected (outer half).

T3—Tumor invades perivesical (around the bladder) fatty tissue.

T3a—microscopically

T3b—macroscopically (e.g., visible tumor mass on the outer bladder tissue).

T4—Tumor invades prostate, uterus, vagina, pelvic wall or abdominal wall.

T4a—Tumor invades prostate, uterus, vagina.

T4b—Tumor invades pelvic wall or abdominal wall.

Additionally, “grading” is also used to characterize bladder cancer. A pathologist defines the grade of bladder cancer from a bladder biopsy. The current system of grading uses only three different grades: well-differentiated, moderately differentiated, and poorly differentiated (or Grade I, II or III).

The invention will be further described by the following non-limiting examples.

### Example 1

### Pre-Operative Plasma Levels of Transforming Growth Factor β₁ Strongly Predict Clinical Outcome in Patients with Bladder Carcinoma

Materials and Methods

Patient Population

Fifty-one patients who were treated with radical cystectomy and pelvic lymphadenectomy for biopsy-proven muscle invasive or refractory superficial TCC at The Methodist Hospital, Houston, Tex., were studied. There were 47 (92%) males and 4 (8%) females, and the mean age was 65.0±8.5 y (median 67, range 41 to 76) at time of cystectomy. The median follow up was 45.7 months (range 4 to 61) for those patients alive at the time of analysis.

Only two patients had clinical evidence of another secondary cancer prior to cystectomy. One patient underwent a colon resection for colon cancer 10 years prior to cystectomy, and radiation therapy for prostate cancer 13 years prior to cystectomy. The second patient had radiation therapy for prostate cancer 16 years prior to cystectomy. Both patients were free of biochemical and clinical disease recurrence for prostate cancer and colon cancer prior to cystectomy and at the time of last follow-up. No other patient was treated either pre-operatively or post-operatively with radiation therapy. One patient was treated with neoadjuvant MVAC chemotherapy prior to surgery for metastases to regional lymph nodes detected on a previous lymphadenectomy. Twenty-four patients were previously treated with intravesical therapy (IVT) including Bacillus Calmette Guerin, Mitomycin C, and/or Thiotepa. The indications for cystectomy in patients with initial Ta, T1, or Tis bladder TCC included IVT failure (n=6), multiple recurrences (n=5), aggressive histo-pathologic features (high-grade tumors, presence of CIS, multifocal extensive disease) (n=4), or progression to muscle invasive cancer (n=19). Seventeen patients underwent cystectomy for an initial presentation of muscle invasive disease.

Plasma TGF-β₁ levels were also assessed in 44 healthy patients without cancer. This group was composed of three sets of consecutive patients who participated in the Baylor Prostate Center's weekly prostate cancer screening program. They had no prior history of cancer or chronic disease, a normal digital rectal examination, and a PSA of less than 2.0 ng/mL, a PSA range that has an estimated probability of prostate cancer detection of less than 1% in the first 4 years after screening (Smith et al., 1996).

TGF-β₁ Measurements

Previously, the effect of the collection format on TGF-β₁ levels was assessed by comparing TGF-β₁ levels in VACUTAINER CPT citrate plasma, VACUTAINER K₃ EDTA plasma, and VACUTAINER BrandSST serum (Becton Dickinson VACUTAINER Systems, Franklin Lakes, N.J.) from synchronously drawn blood specimens of 10 consecutive, healthy screening patients (Shariat et al., 2001c). TGF-β₁ levels were found to be 3 to 6-times higher when measured in serum than when measured in platelet-poor plasma. Plasma from VACUTAINER CPT sodium citrate tubes was used for TGF-β₁ measurements. Plasma TGF-β₁ levels were lower when collected in VACUTAINER CPT citrate plasma compared to VACUTAINER K₃ EDTA plasma because the top plasma layer is diluted primarily by 1.0 mL, of 0.1 mol/L sodium citrate anticoagulant.

Plasma samples were collected on an ambulatory basis on the morning of the scheduled day of surgery after a preoperative overnight fast. Blood was collected into VACUTAINER CPT 8 mL tubes containing 0.1 mL of 1 M sodium citrate anticoagulant and centrifuged at room temperature for 20 minutes at 1500 g. The top layer corresponding to plasma was decanted using sterile transfer pipettes and immediately frozen and stored at −80° C. in polypropylene cryopreservation vials (Nalgene, Nalge Nunc International, Rochester, N.Y.). Prior to assessment, an additional centrifugation step of the plasma was performed at 10,000 g for 10 minutes at room temperature for complete platelet removal. For quantitative measurements of plasma TGF-β₁ levels, a quantitative sandwich enzyme immunoassay was used (QUANTIKINE Human TGF-β₁ Elisa kit, R&D Systems, Minneapolis, Minn.) that is specific for TGF-β₁ and does not cross-react with TGF-β₂ or TGF-β₃. Recombinant TGF-β₁ was used as a standard. Every sample was run in duplicate, and the mean was used for data analysis. Differences between the two measurements were minimal, as shown the intra-assay precision coefficient of variation of only 6.5±3.1%.

Pathological Examination

All histologic slides were reviewed without knowledge of clinical outcome. The 1997 TNM classification was used for pathologic staging and the WHO classification was used for pathologic grading. Eighteen patients (35%) had clinically occult prostate cancer on their pathological prostate specimens. The level of capsular invasion (PCI), with respect to the stroma of the prostate, prostatic capsule, and periprostatic soft tissue, was classified as described previously by Wheeler et al. (1998).

Post-Operative Follow-Up

Each patient was scheduled to have a physical examination, laboratory tests, and a chest radiograph postoperatively every four months for the first year, semiannually in the second year, and annually thereafter, or as clinically indicated. High-risk patients (extravesical involvement or lymph node metastases) continued a semiannual visit schedule through the fifth year, and annually thereafter. Computed tomography scans of abdomen and pelvis were performed annually for the first two years and as indicated by history and physical exam in subsequent years. Cytologic examination of urethral washings were performed annually. Bone scintigraphy, or brain computed tomography scan were carried out when clinically indicated. Five patients (10%) received adjuvant chemotherapy (MVAC) after surgery for adverse pathologic characteristics including regional, distant lymph node metastases, or extravesical involvement. Eleven patients (22%) received MVAC for disease recurrence. For the patients with concomitant prostate cancer, biochemical progression was defined as a sustained elevation, on 2 or more occasions, of PSA>0.2 ng/mL.

Statistical Analysis

Differences in plasma TGF-β₁ levels were assessed using the Mann-Whitney U test. Spearman's rank correlation coefficient was used to compare age and TGF-β₁ levels. Logistic regression was used for univariate and multivariate analysis of binary outcome variables. The Kaplan-Meier method was used to calculate survival functions, and differences were assessed with the log rank statistic. Multivariate survival analysis was performed with the Cox proportional hazard regression model. Clinical and pathological stage were evaluated as Tis, Ta, and T1 versus>T2; clinical and pathologic tumor grade were evaluated as grade 1 and 2 versus grade 3. Statistical significance in this study was set as P<0.05. All reported P values are two-sided. All analyses were performed with SPSS statistical package (SPSS version 10.0 for Windows).

Results

Correlation of TGF-β₁ Levels with Clinicopathological Characteristics

At operation, the clinical stages were Tis (n=3, 6%), Ta (n=1, 2%), T1 (n=11, 22%), T2 (n=28, 55%), T3 (n=5, 10%) and T4a (n=3, 6%). Pathological stages were pTis (n=2, 4%), pT1 (n=8, 16%), pT2 (n=18, 35%), pT3 (n=14, 27%), pT4a (n=8, 16%), and pT4b (n=1, 2%). The clinical grades were grade 2 (n=6, 12%) and grade 3 (n=45, 88%); and the pathologic grades were grade 2 (n=7, 14%) and grade 3 (n=44, 86%). Fourteen patients (28%) had pelvic nodal involvement below the common iliac bifurcation. Four of the 14 were categorized as having distant metastatic disease due to lymph node involvement above the common iliac bifurcation, in addition to regional lymph node involvement.

Eighteen patients (35%) had clinically occult prostate cancer diagnosed after pathologic review of prostate specimens. Pathological stages of these prostate cancers were T1 (n=7, 39%), T2 (n=10, 56%), and T3a (n=1, 6%). Pathological Gleason scores were three (n=2, 11%), five (n=6, 33%), six (n=8, 44%), and seven (n=2, 11%) with a median Gleason score of 6. Eight patients (44%) had level 0 prostate capsular invasion (PCI) (tumor confined to the prostatic stroma within the boundary of normal prostatic acini), and six patients (33%) had level 1 PCI (tumor confined to prostatic stroma, but outside the boundary of normal prostatic acini). Previously, it was shown that patients with prostate cancer exhibiting level 0 or level 1 PCI have no or a very low risk of recurrence (Wheeler et al., 1998). Three (17%) patients had level 2 PCI (tumor invading into but not completely through the prostatic capsule), and one patient (6%) had level 3 focal PCI (tumor outside the prostate to a depth of less than one high power field on no more than two separate sections). Postoperative follow up with serum PSA levels was available in 12 of the patients with concomitant prostate cancer. With a median time follow-up of 13.3 months (range, 6 to 23), none of these patients had experienced biochemical recurrence.

Correlation of pre-operative plasma TGF-β₁ levels with various clinicopathological features is shown below in Table 1. Pre-operative plasma TGF-β₁ levels were significantly elevated in patients with pathologic muscle-invasive TCC (P<0.001), lymphovascular invasion (P<0.001), regional lymph node involvement (P<0.001), and distant lymph node involvement (P=0.001). There were no significant differences in TGF-β₁ levels between males and females (6.1±2.7 ng/nL and 6.8±2.1 ng/mL, respectively, P=0.377), between patients who received IVT prior to cystectomy and those who did not (5.9±2.6 ng/mL and 6.3+2.7 ng/mL, respectively, P=0.776), and between patients who had concomitant prostate cancer and those who did not (5.8±2.1 ng/mL and 6.1±2.7 ng/mL, respectively, P=0.279). Furthermore, pretreatment TGF-β₁ levels did not correlate with age at time of cystectomy (P=0.888).

TABLE 1
Pre-operative Plasma TGF-β₁ Levels in Healthy Controls
and Patients Undergoing Radical Cystectomy for Transitional
Cell Carcinoma of the Urinary Bladder
TGF-β₁ (ng/mL)

| | No. of | | | |
|---|---|---|---|---|
| | Patients | Median | Range | P* |
| Healthy Controls | 44 | 4.5 | 1.0 - 6.6 | |
| Bladder Cancer | | | | |
| Pathologic Stage | | | | |
| Ta, Tis, T1 | 10 | 4.5 | 2.5-5.0 | <0.001 |
| ≧T2a | 41 | 5.7 | 3.0-12.7 | |
| Pathologic Grade | | | | |
| 2 | 7 | 4.9 | 2.5-5.5 | 0.130 |
| 3 | 44 | 5.2 | 2.6-12.7 | |
| Carcinoma in situ | | | | |
| Negative | 25 | 5.2 | 2.5-12.7 | 0.714 |
| Positive | 26 | 6.3 | 2.9-12.3 | |
| Lymphovascular Invasion | | | | |
| Negative | 32 | 4.9 | 2.5-10.1 | <0.001 |
| Positive | 19 | 7.9 | 3.6-12.7 | |
| Regional Lymph Node | | | | |
| Metastases | | | | |
| Negative | 37 | 4.9 | 2.5-8.5 | <0.001 |
| Positive | 14 | 9.4 | 4.3-12.7 | |
| Distant Lymph Node | | | | |
| Metastases** | | | | |
| Negative | 47 | 5.0 | 2.5-12.3 | 0.001 |
| Positive | 4 | 11.9 | 10.9-12.7 | |

*Mann-Whitney U test
**Above the common iliac bifurcation.

In both univariate and multivariate logistic regression analyses that included pre-operative TGF-β₁ level, clinical stage, and clinical grade, pre-operative plasma TGF-β₁ was a significant predictor of lymphovascular invasion (P=0.002; Hazard ratio 1.895, 95% CI 1.261-2.846) and lymph node involvement (P=0.030; Hazard ratio 12.826, 95% CI 1.359-38.522). In univariate logistic regression analysis, pre-operative plasma TGF-⊖₁ level was also a significant predictor of invasive pathologic stage (P=0.011, Hazard ratio 3.685, 95% CI 1.353-10.037), but failed to retain its predictive value for invasion in multivariate analysis that included pre-operative TGF-β₁ level (P=0.173; Hazard ratio 1.665, 95% CI 0.263-20.548), clinical stage, and clinical grade. TGF-β₁ Association with Bladder Cancer Stage Mean TGF-β₁ levels in the 44 healthy men was 4.5 +1.2 ng/mL (median 4.70, range 1.0-6.6). The mean plasma TGF-β₁ level in patients with regional and distant lymph node metastases was significantly higher than that in patients without bladder cancer metastases and in healthy subjects (P values<0.001). TGF-β₁ levels were not different between patients without metastases and healthy controls (P=0.093). However, the mean plasma TGF-β₁ level in patients with clinically muscle invasive TCC was significantly higher than that in patients with clinically Tis, Ta, T1 disease (P=0.042) and in healthy subjects (P=0.050), which were not different from each other (P=0.457).

Clinical Outcome as a Function of TGF-β₁ and Other Parameters

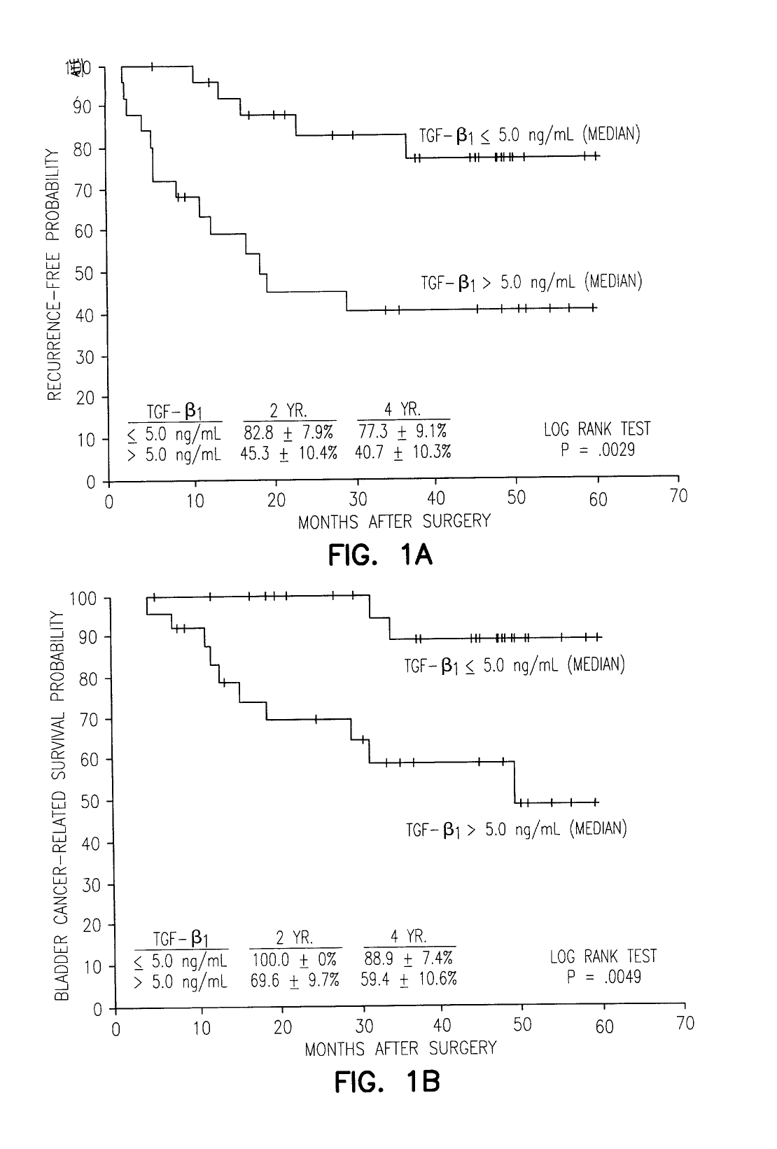

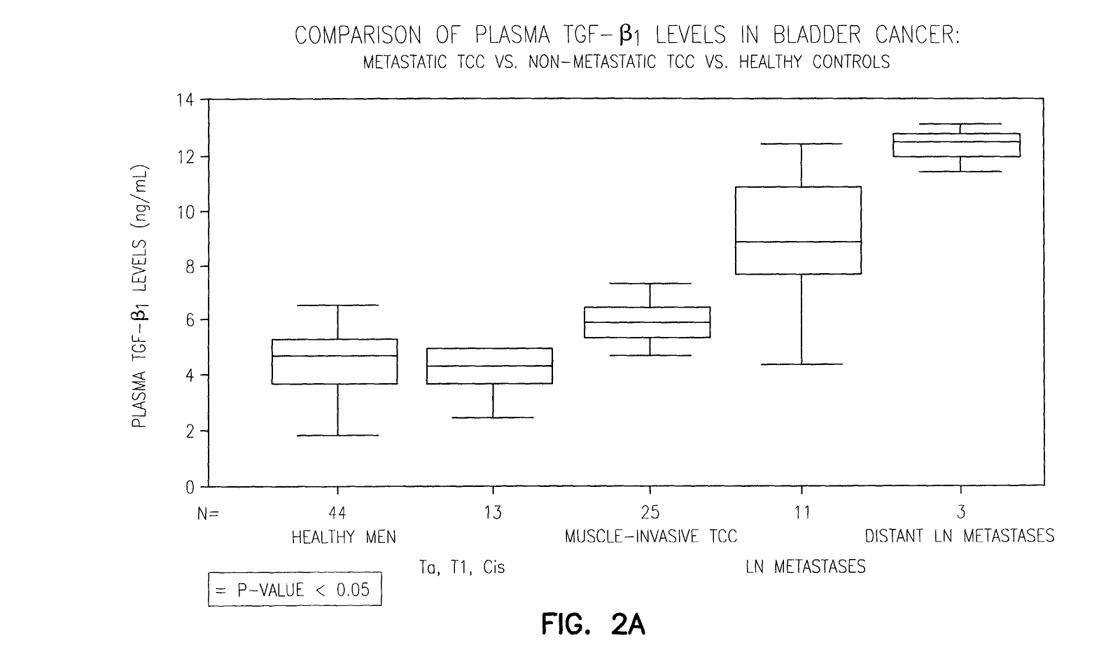

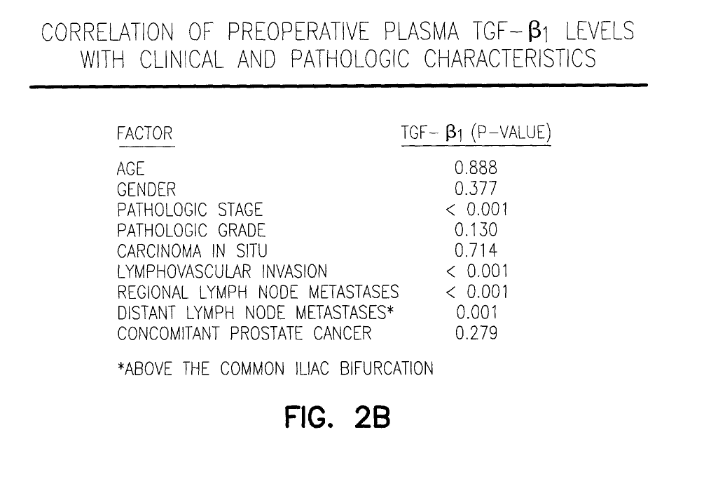

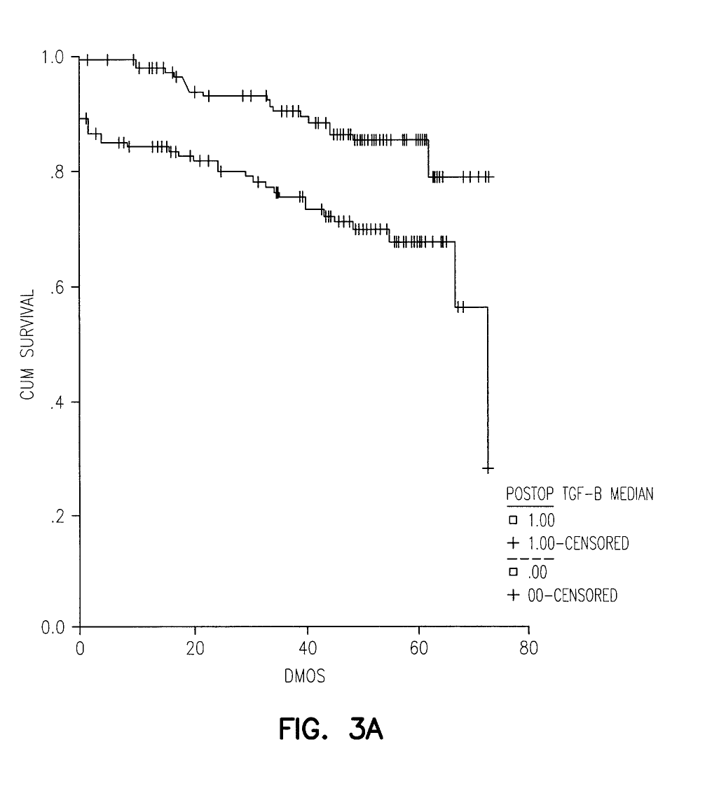

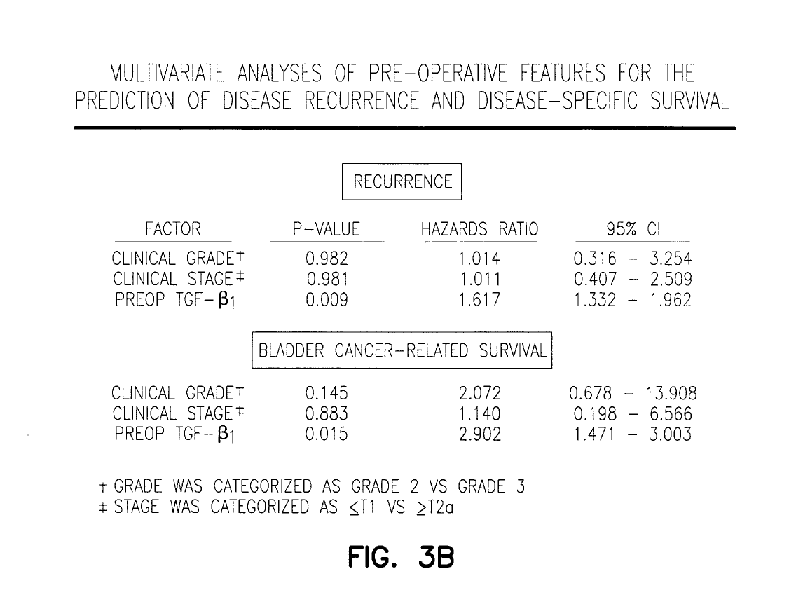

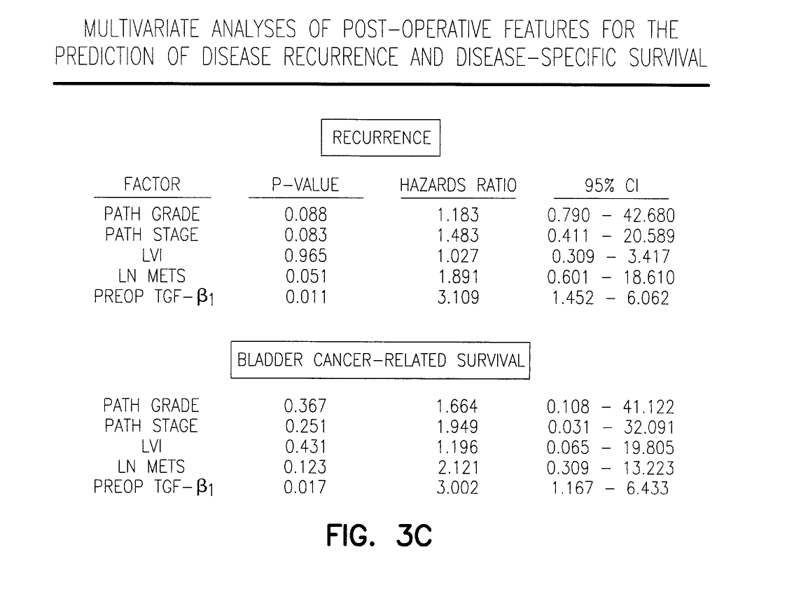

Of 51 patients, 19 patients (37%) recurred, and 19 patients (37%) were dead at the time of analysis. While 12 patients (63%) died of metastatic bladder cancer, 7 (37%) died of other causes without evidence of disease recurrence at the time of death. The overall recurrence-free survival was 59.2±7.3 (SE) at 3 years and at 5 years; the overall disease-specific survival was 74.3±6.8 (SE) at 3 years and 68.4±8.5 (SE) at 5 years. Using the log rank test, patients with plasma TGF-β₁ levels above the median (5.0 ng/mL) were found to have a significantly increased probability of recurrence (P=0.0029; FIG. 1A) and an increased probability of bladder-cancer specific mortality (P=0.0049, FIG. 1B). In the subgroup of patients with pathologic muscle invasive TCC (median follow-up 45.5 months, range 6 to 59.7), patients with TGF-β₁ levels above the median (5.5 ng/mL) had a significantly higher risk of disease recurrence (P=0.0046) and of bladder-cancer specific mortality (P=0.0013).

On univariate Cox proportional hazards regression analyses, plasma TGF-β₁ was associated with the risk of recurrence (P<0.001, Hazard ratio 1.619, 95% CI 1.351-1.939) and of bladder cancer-related death (P<0.001, Hazard ratio 1.903, 95% CI 1.419-2.553). In a pre-operative multivariate model that included pretreatment TGF-β₁ level, clinical stage, and clinical grade, plasma TGF-β₁ level was an independent predictor of disease recurrence (P=0.009) and disease-specific survival (P=0.015) (Table 2). Similarly, in a post-operative multivariate model that included pretreatment TGF-β₁ level, pathological stage, pathological grade, lymphovascular invasion, and lymph node status, plasma TGF-β₁ level was independently associated with disease recurrence (P=0.011) and bladder cancer related death (P=0.017).

TABLE 2
Multivariate Cox Regression Analysis of Pre-operative and
Post-operative Features for the Prediction of Disease
Recurrence and Disease-specific Survival of 51 Patients
Treated with Radical Cystectomy for Transitional
Cell Carcinoma of the Urinary Bladder

| | Pre-operative Features | | | | | |
|---|---|---|---|---|---|---|
| | Recurrence | | | Disease specific survival | | |
| | Hazard | | | Hazard | | |
| | ratio | P | 95% CI | ratio | P | 95% CI |
| Clinical | 1.014 | 0.982 | 0.316-3.254 | 2.072 | 0.145 | 0.678-13.908 |
| grade* | | | | | | |
| Clinical | 1.011 | 0.981 | 0.407-2.509 | 1.140 | 0.883 | 0.198-6.566 |
| stage** | | | | | | |
| Preoperative | 1.617 | 0.009 | 1.332-1.962 | 2.902 | 0.015 | 1.471-3.003 |
| TGF-β₁ | | | | | | |
| levels | | | | | | |
| Pathologic | 1.083 | 0.088 | 0.790-42.680 | 1.664 | 0.367 | 0.108-41.122 |
| grade* | | | | | | |
| Pathologic | 1.883 | 0.083 | 0.411-20.589 | 1.949 | 0.351 | 0.031-32.091 |
| stage** | | | | | | |
| Lympho- | 1.027 | 0.965 | 0.309-3.417 | 1.196 | 0.431 | 0.065-19.805 |
| vascular | | | | | | |
| invasion | | | | | | |
| Lymph | 1.891 | 0.060 | 0.601-18.610 | 2.021 | 0.323 | 0.309-13.223 |
| node | | | | | | |
| metastases | | | | | | |
| Preoperative | 3.109 | 0.011 | 1.452-6.062 | 3.002 | 0.017 | 1.167-6.433 |
| TGF-β₁ | | | | | | |
| levels | | | | | | |

*Clinical and pathological tumor grades were analyzed as dichotomous variables (grade 1, 2 versus grade 3)
**Clinical and pathological tumor stages were analyzed as dichotomous variables (≦Ti versus ≧T2a).

Discussion

TGF-β₁ levels were elevated in patients with clinically invasive TCC. Furthermore, it was found that patients with regional and distant lymph node metastases had significantly higher TGF-β₁ levels than those without metastases. A significant association was found between pre-operative plasma TGF-β₁ levels and established markers of biologically aggressive bladder cancer, such as pathologic stage, lymphovascular invasion, and lymph node metastases in patients undergoing radical cystectomy. Pre-operative plasma TGF-β₁ level was found to be a powerful independent predictor of lymph node metastases, lymphovascular invasion, disease recurrence, and bladder cancer-related death in cystectomy patients.

In pre-operative multivariate analyses, TGF-β₁ level was an independent predictor of lymphovascular invasion and lymph node metastases. An association between elevated TGF-β₁ levels and locally advanced bladder cancer has been reported by Eder et al. (1996). In concordance with Eder et al., the present study found that patients with advanced pathological stage had higher TGF-β₁ levels than patients with superficial disease and healthy controls (at least a 49% and 63%, respectively), and that TGF-β₁ levels in patients with superficial bladder cancer were not significantly different from those in healthy patients, a finding that limits the potential value of TGF-β₁ as a diagnostic tool for detection of bladder cancer.

TGF-β₁ levels in patients with bladder cancer metastatic to regional and distant lymph nodes were found to be markedly elevated. The majority of patients with organ confined disease and negative nodes, whose local tumor has been completely removed, are reported to have a favorable cancer-specific survival with 5-year actuarial survival rates between 76% and 89% (Amling et al., 1994; Hautmann and Paiss, 1998). However, approximately 70% of patients with lymph node involvement eventually fail local therapy and develop distant metastases that lead to death, regardless of earlier success in eradicating the local disease (Lerner et al., 1993). Thus, while the pT category of the primary tumor is a substantial stratification variable in determining who may or may not benefit from radical surgery, the nodal tumor burden is the most significant pathologic prognostic factor in patients with bladder TCC. In contrast to the findings herein, Eder et al. (1996) failed to detect a significant elevation in patients with regional nodal metastases. However, pathologic staging of lymph nodes was performed in a small group of patients (19 of their initial 57 patients) using mean values with a wide range of TGF-β₁ levels. Finally, Eder et al. relied on serum samples, which are known to be less reliable than plasma samples for measuring TGF-β₁ levels. Since TGF-β₁ is present in platelet granules and is released upon platelet activation, TGF-β₁ in serum is likely to be derived at least partly from damaged platelets. To ensure complete platelet removal, an additional centrifugation, as well as using plasma samples, was performed.

Elevated plasma TGF-β₁ levels had the highest hazard ratio for the prediction of established lymph node metastases (Hazard ratio 12.8). Furthermore, the association of TGF-β₁ levels with disease recurrence and disease-specific mortality, even in patients with effective local control of their disease, suggests that TGF-β₁ levels are primarily associated with metastases, including occult metastases, that increase the risk of disease progression and bladder cancer-related death. A similar association between pre-operative TGF-β₁ levels and clinical and occult metastatic disease in patients with prostate cancer undergoing radical prostatectomy was reported by Shariat et al. (2001c). These data suggest that circulating TGF-β₁ levels are fundamentally associated with the process of metastasis, either directly due to tumor factors or due to a host response that is measurable at the earliest stages of this process.

While significantly elevated levels of TGF-β₁ in patients with lymph node metastases was found, there was an overlap between TGF-β₁ levels in patients with metastatic disease and those in patients with either Tis, Ta, or T1, or locally advanced bladder cancer. Because of the overlap, no single cut-off level could be used to classify patients with regard to bladder cancer stage. Nevertheless, the TGF-β₁ level was the strongest independent predictor of lymph node metastases, disease recurrence, and disease-specific survival. A nomogram that includes TGF-β₁ together with other significant predictors would be a useful model as it would proportionally assign the predictive value of TGF-β₁.

Eighteen patients had concomitant adenocarcinoma of the prostate on their prostate specimen. It has been shown that TGF-β₁ levels are elevated in patients with advanced stage prostate cancer and in patients with metastatic prostate cancer (Kong et al., 1999; Adler et al., 1999; Ivanovic et al., 1995; Kakehi et al., 1996; Shariat et al., 2001c). However, no difference was found in plasma TGF-β₁ levels between patients with pathologically localized prostate cancer and healthy men without cancer (Shariat et al., 2001c). In the present study, 94% of the men with concomitant prostate cancer had pathologically localized prostate cancer, 89% had Gleason score below or equal to six, 78% had tumor confined to the prostatic stroma, and 94% had no extracapsular disease. The pathologic stage, the Gleason score, and the level and extent of prostatic capsular invasion are known to be strong predictors of progression after radical prostatectomy (Wheeler et al., 1998; Eastham et al., 2000; Catalona et al., 1998). In addition, none of the patients in this study experienced recurrence of prostate cancer.

Conclusions

Plasma TGF-β₁ levels are markedly elevated in patients with bladder cancer metastatic to regional and distant lymph nodes. Moreover, pre-operative plasma TGF-β₁ level is a strong predictor of disease recurrence and bladder cancer-related survival after surgery. Thus, pretreatment plasma TGF-β₁ levels may be useful as a staging and prognostic marker for patients undergoing local therapy such as cystectomy or radiation therapy for bladder TCC.

### EXAMPLE 2

### Pre-Operative Plasma Levels of Interleukin-6 and its Soluble Receptor Predict Disease Recurrence and Survival in Patients with Bladder Cancer

Materials and Methods

Patient Population

Pre-operative plasma levels of IL-6 and IL-6sR were measured in 51 patients who were treated with radical cystectomy and pelvic lymphadenectomy for biopsy-proven muscle invasive or refractory superficial TCC. There were 47 (92%) males and 4 (8%) females, and the mean age was 65.0±8.5 years (median 67, range 41 to 76) at the time of cystectomy. The median follow-up was 45.7 months (range 4 to 61) for those patients alive at the time of analysis.

Only two patients had clinical evidence of another secondary cancer prior to cystectomy. One had received radiation therapy for prostate cancer 13 years earlier and had undergone resection for colon cancer 10 years earlier. The second patient had received radiation therapy for prostate cancer 16 years before the cystectomy. Both patients were free of biochemical and clinical disease recurrence of prostate cancer and colon cancer at the time of cystectomy and last follow-up. No other patient was treated either pre-operatively or post-operatively with radiation therapy. One patient was treated with neoadjuvant chemotherapy prior to surgery for metastases to regional lymph nodes detected on a previous lymphadenectomy. Twenty-four patients were previously treated with intravesical therapy (IVT) including Bacillus Calmette Guerin (BCG), Mitomycin C, and/or Thiotepa. A clinicopathological profile of the patient population is shown in Table 3. The indications for cystectomy in patients with initial Ta, T1, or Tis bladder TCC included IVT failure (n=6), multiple recurrences (n=5), aggressive histopathologic features (high-grade tumors, presence of CIS, multifocal extensive disease) (n=4), or progression to muscle invasive cancer (n=19). Seventeen patients underwent cystectomy for an initial presentation of muscle invasive disease.

TABLE 3
Pre-operative Plasma IL-6 and IL-6 Soluble Receptor Levels in
Healthy Controls and Patients Undergoing Radical Cystectomy for
Transitional Cell Carcinoma of the Urinary Bladder

| | | IL-6 | | | IL-6sR | | |
|---|---|---|---|---|---|---|---|
| | No. of | Median | Range | | Median | Range | |
| | Patients | (pg/mL) | (pg/mL) | P* | (ng/mL) | (ng/mL) | P** |
| Healthy Controls | 44 | 1.41 | 0.54-3.52 | | 19.75 | 10.10-30.80 | |
| Bladder Cancer | 51 | 4.80 | 1.02-7.44 | <0.001 | 21.23 | 13.04-32.16 | 0.333 |
| Clinical Stage | | | | | | | |
| Superficial | 15 | 4.07 | 1.52-6.76 | 0.047 | 18.86 | 14.77-28.80 | 0.052 |
| Tis | 3 | | | | | | |
| Ta | 1 | | | | | | |
| T1 | 11 | | | | | | |
| Invasive | 36 | 5.22 | 1.02-7.44 | | 22.26 | 13.04-32.16 | |
| T2 | 28 | | | | | | |
| T3 | 5 | | | | | | |
| T4a | 3 | | | | | | |
| Clinical Grade | | | | | | | |
| 2 | 6 | 4.87 | 3.29-6.76 | 0.558 | 20.14 | 14.77-28.80 | 0.755 |
| 3 | 45 | 4.80 | 1.02-7.44 | | 21.38 | 13.04-32.16 | |
| Pathologic Stage | | | | | | | |
| Superficial | 10 | 3.31 | 1.52-6.76 | 0.044 | 18.41 | 14.77-21.45 | 0.036 |
| pTis | 2 | | | | | | |
| pT1 | 8 | | | | | | |
| Invasive | 41 | 5.14 | 1.02-7.44 | | 22.01 | 13.04-32.16 | |
| pT2 | 18 | | | | | | |
| pT3 | 14 | | | | | | |
| pT4a | 8 | | | | | | |
| pT4b | 1 | | | | | | |
| Pathologic Grade | | | | | | | |
| 2 | 7 | 5.37 | 3.29-6.76 | 0.546 | 18.86 | 14.77-21.55 | 0.043 |
| 3 | 44 | 4.77 | 1.02-7.44 | | 21.58 | 13.04-32.16 | |
| Lymphovascular | | | | | | | |
| Invasion | | | | | | | |
| Negative | 32 | 4.13 | 1.02-7.44 | 0.044 | 20.24 | 13.04-30.15 | 0.034 |
| Positive | 19 | 5.46 | 3.28-6.56 | | 25.23 | 15.34-32.16 | |
| Regional Lymph | | | | | | | |
| Node Metastases | | | | | | | |
| Negative | 37 | 4.19 | 1.02-6.76 | 0.025 | 20.32 | 13.04-30.83 | 0.008 |
| Positive | 14 | 5.73 | 2.01-7.44 | | 26.07 | 15.05-32.16 | |
| Distant Lymph | | | | | | | |
| Node | | | | | | | |
| Metastases*** | | | | | | | |
| Negative | 47 | 4.72 | 1.02-7.44 | 0.047 | 20.89 | 13.04-30.83 | 0.002 |
| Positive | 4 | 6.00 | 5.33-6.56 | | 209.85 | 28.84-32.16 | |
| Sex | | | | | | | |
| Male | 47 | 4.83 | 1.02-7.4 | 1.00 | 20.90 | 13.04-32.16 | 0.713 |
| Female | 4 | 4.46 | 3.38-5.97 | | 23.37 | 16.54-25.57 | |
| Prostate Cancer | | | | | | | |
| Negative | 33 | 5.23 | 2.01-7.44 | 0.152 | 21.14 | 14.77-32.16 | 0.379 |
| Positive | 18 | 4.73 | 1.02-5.79 | | 21.23 | 13.04-30.83 | |

*Mann-Whitney U test
**Independent sample T-test
***Above the common iliac bifurcation

Plasma IL-6 and IL-6sR levels in 44 healthy patients without cancer were also assessed. This group was composed of three sets of consecutive patients who participated in a prostate cancer screening program. They had no prior history of cancer or chronic disease, a normal digital rectal examination, and a PSA of less than 2.0 ng/mL. This PSA range has an estimated probability of prostate cancer detection of less than 1% in the first 4 years after screening (Smith et al., 1996).

Pathological Examination

All histologic slides were reviewed without knowledge of clinical outcome. The 1997 TNM classification was used for clinical and pathologic staging. The WHO classification was used for clinical and pathologic grading. Pathological stages were T1 (n=7, 39%), T2 (n=10, 56%), and T3a (n=1, 6%). Pathological Gleason scores were three (n=2, 11%), five (n=6, 33%), six (n=8, 44%), and seven (n=2, 11%) with a median Gleason score of 6.

IL-6 and IL-6sR Measurements

Plasma samples were collected on the morning of the scheduled day of surgery after a pre-operative overnight fast. Samples were collected from healthy controls at the time of prostate cancer screening. Blood was collected into VACUTAINER CPT 8 mL tubes containing 0.1 mL of 1 M sodium citrate anticoagulant and centrifuged at room temperature for 20 minutes at 1500 g. The top layer corresponding to plasma was decanted using sterile transfer pipettes and immediately frozen and stored at −80° C. in polypropylene cryopreservation vials (Nalgene, Nalge Nunc International, Rochester, N.Y.). For quantitative measurements of plasma IL-6 and IL-6sR levels, quantitative sandwich enzyme immunoassays (QUANTIKINE Human IL-6 Elisa kit and QUANTIKINE Human IL-6sR Elisa kit, R&D Systems, Minneapolis, Minn.) that are specific for IL-6 and IL-6sR, respectively, were used. Every sample was run in duplicate, and the mean was used for data analysis. Differences between the two measurements for both IL-6 and IL-6sR were minimal, as shown by the intra-assay precision coefficients of variation of only 4.27±3.64% and 3.98±3.31%, respectively.

Post-Operative Treatment

Five patients (10%) received adjuvant chemotherapy with MVAC (methotrexate, vinblastine, doxorubicin, cisplatin) after surgery for adverse pathologic characteristics including regional, distant lymph node metastases, or extravesical involvement. Eleven patients (22%) received MVAC for disease recurrence. For the patients with concomitant prostate cancer, biochemical progression was defined as a sustained elevation, on 2 or more occasions, of PSA>0.2 ng/mL.

Statistical Analysis

Differences in plasma IL-6 and IL-6sR levels were assessed using the Mann-Whitney U test and the independent sample T-test, respectively. Spearman's rank correlation coefficient was used to compare age and cytokine levels. Logistic regression was used for univariate and multivariate analyses of binary outcome variables. The Kaplan-Meier method was used to calculate survival functions, and differences were assessed with the log rank statistic. Multivariate survival analysis was performed with the Cox proportional hazard regression model. Clinical and pathological stage were evaluated as Tis, Ta, and T1 versus≧T2; clinical and pathologic tumor grades were evaluated as grade 2 versus grade 3. Statistical significance in this study was set as P<0.05. All reported P values are two-sided. All analyses were performed with SPSS statistical package (SPSS version 10.0 for Windows).

Results

Comparison with Healthy Controls

Mean plasma IL-6 level in the 44 healthy men was 1.51±0.61 pg/mL (median 1.41, range 0.54-3.52). The mean IL-6 level in bladder cancer patients (4.51±1.48 pg/mL, median 4.80, range 1.02-7.44) was significantly higher than in the healthy controls (P<0.001). However, there was no significant difference in mean IL-6sR levels between healthy men (20.3 ng/mL, median 19.75, range 10.1-30.8) and bladder cancer patients (21.3 ng/mL, median 21.2, range 13.0-32.2) (P=0.333).

Correlation of IL-6 and IL-6 Soluble Receptor Levels with Clinicopathological Characteristics

Correlation of pre-operative plasma IL-6 and IL-6sR levels with various clinicopathological features is shown in Table 4. IL-6 and IL-6sR levels were elevated in patients with pathologic muscle invasive TCC(P=0.044 and P=0.036, respectively), lymphovascular invasion (P=0.044 and P=0.034, respectively), regional lymph node involvement (P=0.025 and P=0.008, respectively), and distant lymph node involvement (P=0.047 and P=0.002, respectively). Additionally, levels of IL-6sR (P=0.043), but not IL-6 (P=0.546), were elevated in patients with pathologic grade 3 tumors versus grade 2. Increased levels of IL-6 were associated with advanced clinical stage (P=0.047), but neither IL-6 nor its receptor was associated with clinical grade. There were no significant differences in IL-6 or IL-6sR levels between males and females or between patients with and without pathologically detected concomitant prostate cancer. Furthermore, while pretreatment levels of IL-6 and IL-6sR did not correlate with age at the time of cystectomy (P=0.179 and P=0.942, respectively), IL-6 and IL-6sR did correlate with one another (P=0.043, r=0.373).

In both univariate and multivariate logistic regression analyses that included pre-operative IL-6, clinical stage, and clinical grade, pre-operative plasma IL-6 was an independent predictor of lymphovascular invasion (P=0.050; Hazard ratio 1.666, 95% CI 1.048-2.585) and regional lymph node metastases (P=0.008; Hazard ratio 2.856, 95% CI 1.318-6.186). In an alternative model including IL-6sR instead of IL-6, IL-6sR was also independently associated with lymphovascular invasion (P=0.021; Hazard ratio 1.878, 95% CI 1.025-3.354) and lymph node metastases (P=0.007; Hazard ratio 1.756, 95% 1.065-2.806). In multivariate logistic regression analyses that included IL-6, IL-6sR, clinical stage, and clinical grade, both cytokines were independent predictors of lymph node metastases (IL-6: P=0.018, Hazard ratio 2.846, 95% CI 1.199-6.752; IL-6sR: P=0.019, Hazard ratio 2.243, 95% CI 1.037-4.491), but only IL-6sR maintained predictive value for lymphovascular invasion (P=0.046; Hazard ratio 1.557, 95% CI 1.003-2.336). In univariate logistic regression models, neither IL-6 nor IL-6sR levels were predictive of final pathologic stage (P=0.079 and P=0.112, respectively).

Clinical Outcome as a Function of IL-6, IL-6sR, and Other Parameters

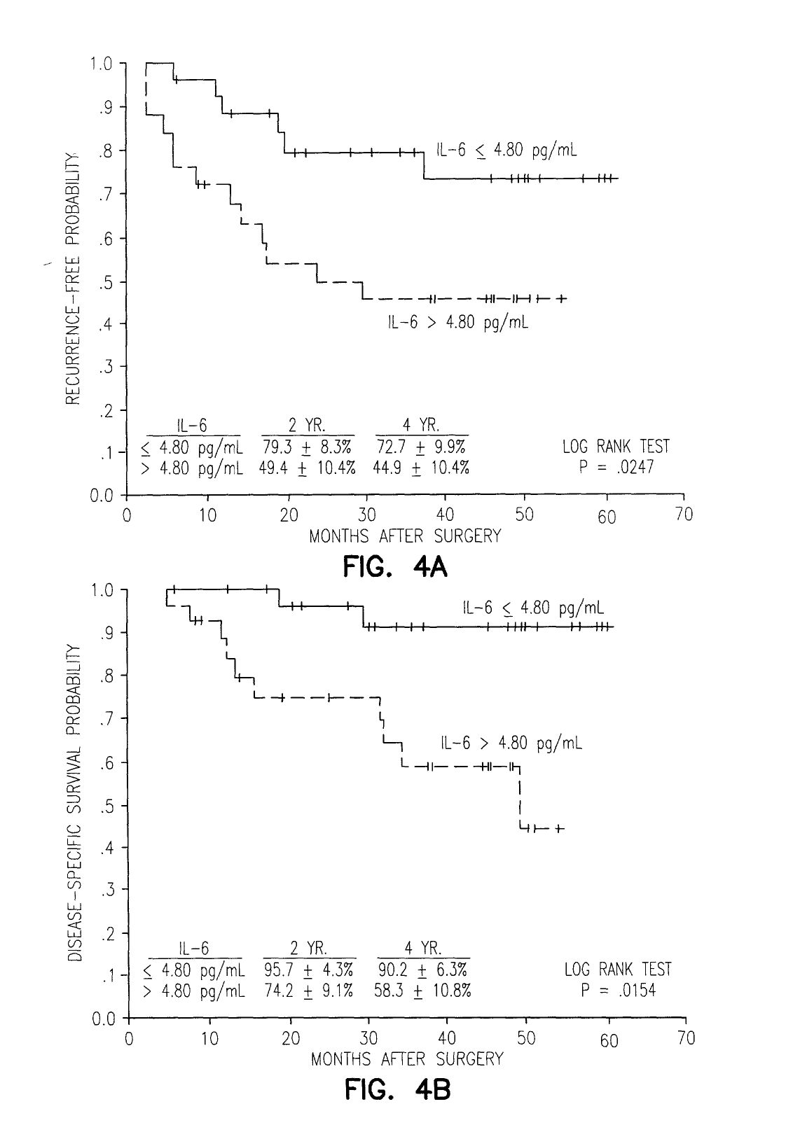

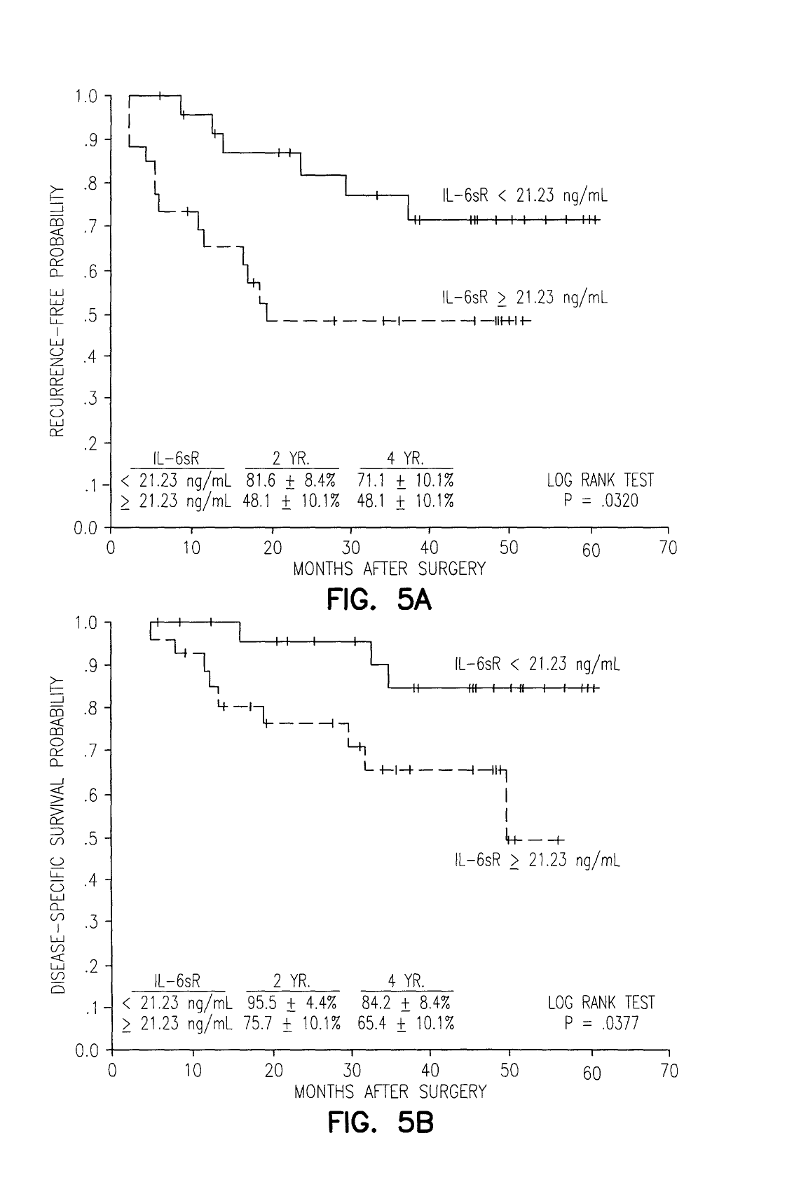

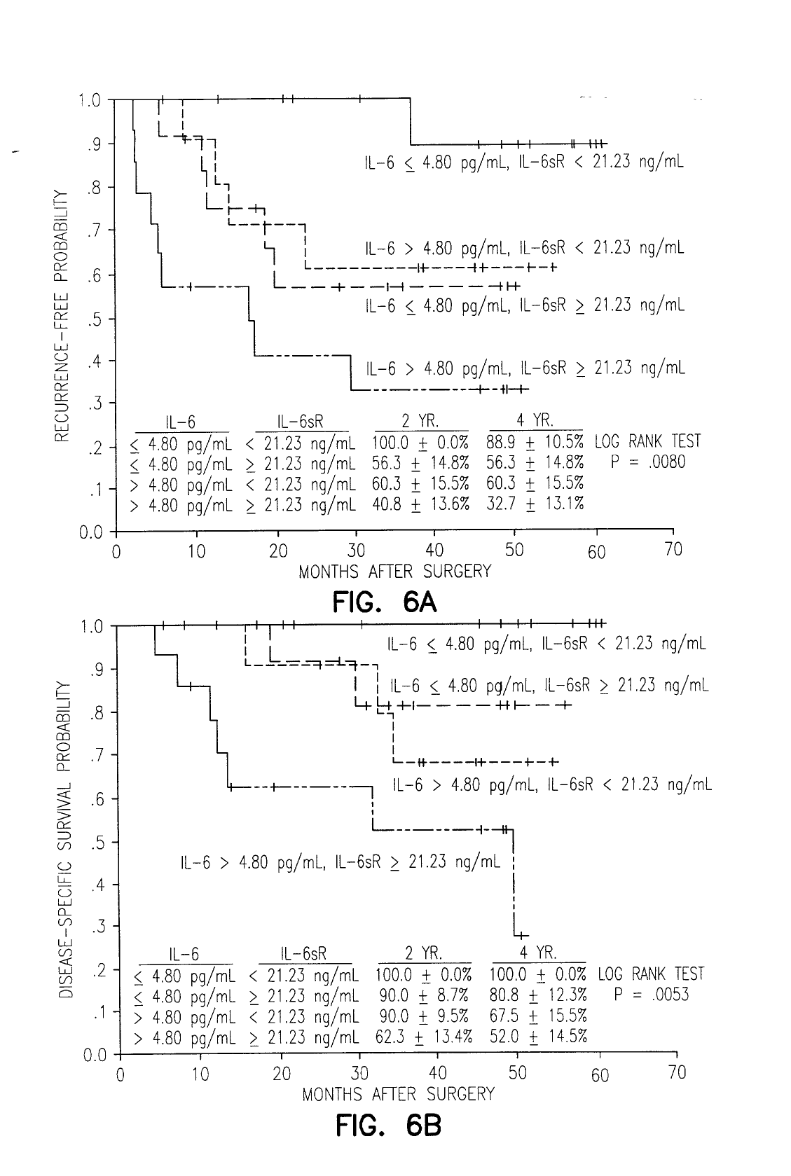

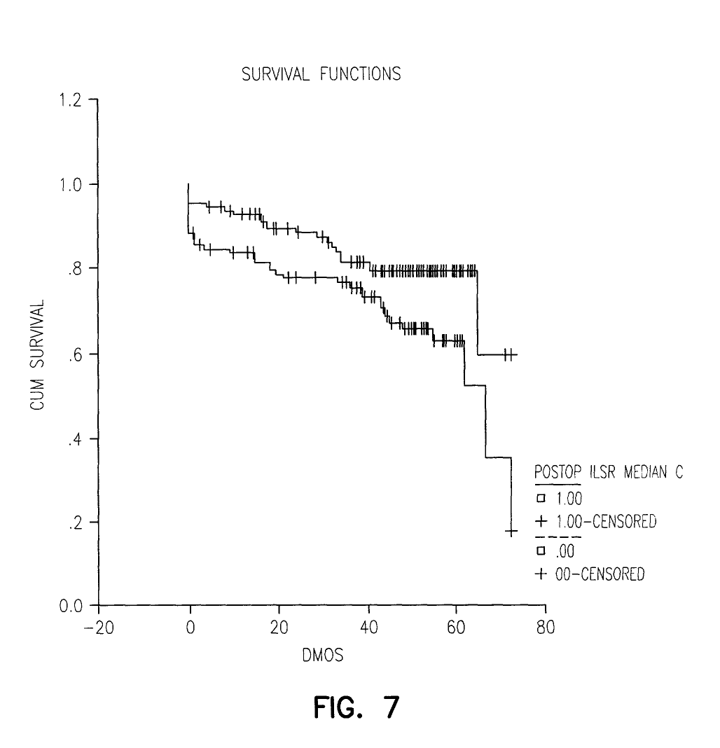

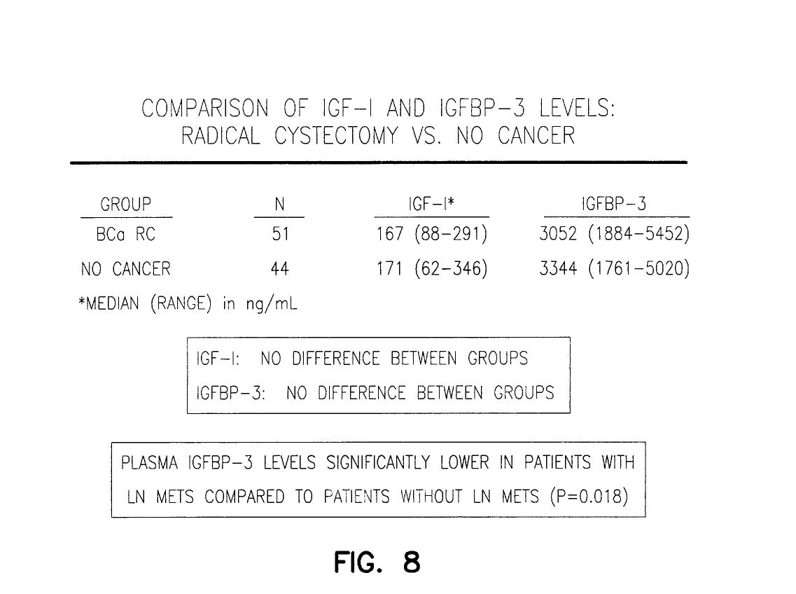

Of 51 patients, 19 (37%) had recurrence, and 19 (37%) were dead at the time of analysis. While 12 (63%) of 19 patients died from metastatic bladder cancer, 7 (37%) died of other causes without evidence of disease recurrence at the time of death. The overall recurrence-free survival probability was 64.5±7.0% (SE) at 2 years and 59.2±7.3% (SE) at 4 years; the overall disease-specific survival was 85.2±5.2% (SE) at 2 years and 74.3±6.8% (SE) at 4 years. Using the log rank test, patients with plasma IL-6 levels above the median (4.80 pg/mL) were found to have a significantly increased probability of disease recurrence (P=0.025; FIG. 4A) and bladder cancer-specific mortality (P=0.015; FIG. 4B). The same was true of IL-6sR (median, 21.23 ng/mL) for both recurrence (P=0.032; FIG. 5A) and disease-specific mortality (P=0.038; FIG. 5B). Patients with elevated levels of both IL-6 and IL-6sR had a greater probability of disease recurrence (P=0.008; FIG. 6A) and cancer-specific mortality (P=0.005; FIG. 6B) than did patients with only one cytokine level above the median or both levels below the median.

In univariate Cox proportional hazards regression analyses, plasma IL-6 and IL-6sR were each associated with disease recurrence (P=0.005; Hazard ratio 1.747, 95% CI 1.186-2.573 and P<0.001; Hazard ratio 2.287, 95% CI 1.144-3.449, respectively) and bladder cancer-related mortality (P=0.001; Hazard ratio 2.510, 95% CI 1.454-4.333 and P=0.003; Hazard ratio 2.235, 95% CI 1.074-3.420, respectively). A pre-operative multivariate model that included IL-6, clinical stage, and clinical grade showed that IL-6 was an independent predictor of disease recurrence (P=0.027; Hazard ratio 1.662, 95% CI 1.054-2.374) and disease-specific death (P=0.016; Hazard ratio 2.261, 95% CI 1.118-3.422). In another model that included IL-6sR, clinical stage, and clinical grade, IL-6sR also predicted recurrence (P=0.016; Hazard ratio 2.261, 95% CI 1.118-3.422) and bladder cancer-specific survival (P=0.027; Hazard ratio 2.420, 95% CI 1.057-3.409). In a Cox proportional hazards regression analysis including both IL-6 and IL-6sR along with clinical stage and grade, both IL-6 and the soluble receptor predicted disease-specific survival (P=0.050 and P=0.042, respectively), but IL-6sR was the sole independent predictor of cancer recurrence (P=0.035) (Table 5). Furthermore, a post-operative multivariate model that included pretreatment IL-6sR level, and pathologic grade, pathologic stage, lymphovascular invasion, and lymph node metastases showed that IL-6sR was an independent predictor of cancer recurrence (P=0.034) but not disease-specific survival (P=0.051). In a similar post-operative model, IL-6 failed to retain predictive significance for disease recurrence (P=0.056) or bladder cancer-related death (P=0.054). When IL-6 was added to the model, none of the variables maintained significance.

TABLE 4
Multivariate Cox Regression Analysis of Pre-operative and
Post-operative Features for the Prediction of Disease
Recurrence and Disease-specific Survival of 51
Patients Treated with Radical Cystectomy for
Transitional Cell Carcinoma of the Urinary Bladder

| Recurrence | | | Disease-specific survival | | |
|---|---|---|---|---|---|
| Hazard | | | Hazard | | |
| ratio | P | 95% CI | ratio | P | 95% CI |

Pre-operative Features

| | | | | | | |
|---|---|---|---|---|---|---|
| Clinical | 1.431 | 0.134 | 0.594-4.684 | 1.207 | 0.312 | 0.271-5.433 |
| grade* | | | | | | |
| Clinical | 1.133 | 0.336 | 0.035-4.130 | 1.163 | 0.846 | 0.050-6.745 |
| stage† | | | | | | |
| Pre-operative | 1.417 | 0.084 | 0.954-2.107 | 2.171 | 0.050 | 1.288-3.659 |
| IL-6 levels | | | | | | |
| Pre-operative | 2.242 | 0.035 | 1.100-3.402 | 2.214 | 0.042 | 1.042-3.415 |
| IL-6 sR levels | | | | | | |

Post-operative Features

| | | | | | | |
|---|---|---|---|---|---|---|
| Pathologic | 1.676 | 0.089 | 0.491-38.750 | 1.355 | 0.319 | 0.138-16.143 |
| grade* | | | | | | |
| Pathologic | 1.802 | 0.081 | 0.223-14.553 | 1.657 | 0.208 | 0.238-15.146 |
| stage† | | | | | | |
| Lymphovascular | 1.113 | 0.344 | 0.192-10.072 | 1.406 | 0.310 | 0.201-15.111 |
| invasion | | | | | | |
| Lymph node | 1.948 | 0.063 | 0.734-10.287 | 2.306 | 0.056 | 0.946-28.916 |
| metastases | | | | | | |
| Pre-operative | 2.485 | 0.034 | 1.106-5.493 | 2.613 | 0.051 | 0.954-4.337 |
| IL-6sR levels | | | | | | |

*Clinical and pathological tumor grades were analyzed as dichotomous variables (grade 2 versus grade 3).
†Clinical and pathological tumor stages were analyzed as dichotomous variables (≦T1 versus ≧T2a).

Discussion

Studies have shown increased local and circulating levels of IL-6 in bladder cancer patients with advanced pathologic tumor stage (Cardillo et al., 2000; Seguchi et al., 1992). This elevation of circulating IL-6 was confirmed, and increased plasma IL-6sR was also found, in patients with muscle invasive cancer. However, neither cytokine independently predicted pathologic stage in logistic regression models. In concordance with Seguchi et al., no association was found between IL-6 and pathologic tumor grade (1992). In contrast, IL-6sR levels were elevated in patients with high grade tumors. Regardless of the success of radical cystectomy, approximately 70% of patients with lymph node involvement eventually fail local therapy and develop distant metastases that lead to death (Lerner et al., 1993). Thus, while pathologic tumor staging and grading are substantial determinants of the outcome after radical cystectomy, the involvement of the lymph nodes is a stronger pathologic prognostic factor.

Both IL-6 and IL-6sR levels were found to be elevated in patients with bladder cancer metastatic to regional and distant lymph nodes. IL-6 and IL-6sR predicted lymph node metastases in multivariate logistic regression models. In separate multivariate models, IL-6 and IL-6sR also predicted invasion into the lymphovascular system, but when both were considered in the same model, IL-6sR was the sole independent predictor. These findings are consistent with other studies that demonstrated increased circulating levels of IL-6 in patients with metastatic prostate and colorectal cancers (Adler et all, 1999; Akimoto et al., 1998; Ueda et al., 1994; Shariat et al., 2001c).

Previous studies also suggested that elevated IL-6 levels are a significant prognostic factor in prostate, ovarian, and renal cancers (Nakashima et al., 2000; Belluco et al., 2000; Scambia et al., 1995). Both IL-6 and IL-6sR independently predicted disease recurrence and death from bladder cancer. However, when both cytokines were accounted for together, IL-6sR was the sole independent predictor of cancer recurrence. Additionally, when the post-operative features of pathologic stage, pathologic grade, lymphovascular invasion, and lymph node metastasis were taken into account, IL-6 lost its statistical significance, and IL-6sR independently predicted disease recurrence but not disease-specific death.

Viewed in the context of similar observations made for other cancers, the data support a relationship between elevated circulating IL-6 and IL-6sR levels and metastatic cancer. However, the biologic mechanisms linking metastases with the increase in IL-6 and IL-6sR are not clearly understood. Like other classic tumor markers, IL-6 may be synthesized directly by malignant cells and thus reflect local tumor burden. Tamm et al. (1989) demonstrated that exogenous IL-6 increases motility and decreases adherence of breast carcinoma cell lines, and others have proposed IL-6 involvement in establishing autonomous tumors by the induction of angiogenesis (Motro et al., 1990). These data suggest that IL-6 confers a selective advantage for certain disseminated cells to develop into metastatic tumors. The discovery that bladder carcinoma cell lines produce measurably more IL-6 than normal urothelial cells seems to support this hypothesis (Kinoshita et al., 1999). In vitro studies of melanoma and prostate cancer have suggested that IL-6 switches from paracrine growth inhibitor to autocrine growth stimulator during cancer progression (Takenawa et al, 1991; Lu et al., 1993). The timing of this switch could be closely linked to metastasis, as the tumor's endogenous IL-6 production aids in dissemination, reattachment, and autonomous growth.

Unlike in prostate cancer studies, where elevated IL-6 levels have primarily been limited to metastatic patients (Shariat et al., 2001a; Adler et al., 1999), the data showed IL-6 levels in bladder cancer patients were higher than in healthy controls. Elevated IL-6 levels in both non-invasive and invasive cancers was observed. This suggests that IL-6 production may be an early event in bladder cancer and raises the potential for IL-6 as a diagnostic marker. IL-6sR was not elevated in TCC patients compared to healthy men, but because it mediates and augments the IL-6 response through transsignaling, IL-6sR may play a major role in tumor development. Higher levels of IL-6sR were found in patients with high grade tumors. Genetic mutations in the cells may cause increased differential mRNA splicing, or cellular dedifferentiation in these tumors may proteolytically cleave the membrane-bound IL-6 receptors.

Eighteen patients (35%) had concomitant prostate cancer detected at the time of cystectomy. In the present study, 17 (94%) of the 18 men had pathologically localized prostate cancer. Post-operative serum PSA levels were available for 12 of these patients, with a median follow-up time of 13.3 months (range, 6 to 23), and none of the patients had experienced biochemical recurrence. Finally, no association was found between the presence of concomitant prostate cancer and levels of IL-6 or IL-6sR. Thus, it is believed that the presence of concomitant prostate cancer did not have an impact on the findings.

Conclusions

Pre-operative plasma IL-6 levels were elevated in patients with bladder cancer. Among the patients with bladder cancer, IL-6 and IL-6sR levels were elevated in those with metastases to regional and distant lymph nodes. Plasma IL-6sR and IL-6 levels also predicted disease recurrence and bladder cancer-related mortality.

### EXAMPLE 3

### Correlation of Pre-Operative Plasma Level of IGF-I and IGFBP-3 with Pathologic Characteristics and Clinical Outcome in Patients with Bladder Cancer

Materials and Methods

Patient Population

All studies were undertaken with the approval and institutional oversight of the Institutional Review Board for the Protection of Human Subjects at Baylor College of Medicine. Pre-operative plasma levels of IGF-I and IGFBP-3 were measured in 51 patients who were treated with radical cystectomy and pelvic lymphadenectomy for biopsy-proven muscle invasive or refractory superficial TCC. There were 47 (92%) males and 4 (8%) females, and the mean age was 65.0±8.5 years (median 67, range 41 to 76) at time of cystectomy. The median follow-up was 45.7 months (range 4 to 61) for those patients alive at the time of analysis.

Only two patients had clinical evidence of another cancer prior to cystectomy. One had received radiation therapy for prostate cancer 13 years earlier and had undergone resection for colon cancer 10 years earlier. The second patient had received radiation therapy for prostate cancer 16 years before the cystectomy. Both patients were free of biochemical and clinical disease progression of prostate cancer and colon cancer at the time of cystectomy and last follow-up. No other patient was treated either pre-operatively or post-operatively with radiation therapy. One patient was treated with neoadjuvant chemotherapy prior to surgery for metastases to regional lymph nodes detected on a previous lymphadenectomy. Twenty-four patients were previously treated with intravesical therapy (IVT) including Bacillus Calmette Guerin (BCG), Mitomycin C, and/or Thiotepa. A clinicopathological profile of the patient population is shown in Table 5. The indications for cystectomy in patients with initial Ta, T1, or TIS bladder TCC included IVT failure (n=6), multiple recurrences (n=5), aggressive histo-pathologic features (high-grade tumors, presence of CIS, multifocal extensive disease) (n=4), or progression to muscle-invasive cancer (n=19). Seventeen patients underwent cystectomy for an initial presentation of muscle invasive disease.

Plasma IGF-I and IGFBP-3 levels were also assessed in 44 healthy patients without cancer. This group was composed of three sets of consecutive patients who participated in the Baylor Prostate Center's weekly prostate cancer screening program. They had no prior history of cancer or chronic disease, a normal digital rectal examination, and a PSA of less than 2.0 ng/mL. This PSA range has an estimated probability of prostate cancer detection of less than 1% in the first 4 years after screening.

IGF-I and IGFBP-3 Measurements

Pre-operative serum and plasma samples typically were collected on the morning of the day of surgery after an overnight fast. Samples were collected from healthy controls at the time of prostate cancer screening. Blood was collected into VACUTAINER CPT 8 mL tubes containing 0.1 mL of 1 M sodium citrate anticoagulant (Becton Dickinson, Franklin Lakes, N.J.) and centrifuged at room temperature for 20 minutes at 1500× g. The top layer, corresponding to diluted plasma was decanted using sterile transfer pipettes and immediately frozen and stored at −80° C. in polypropylene cryopreservation vials (Nalge Nunc, Rochester, N.Y.). For quantitative measurements of serum and plasma IGF-I and IGFBP-3 levels, the DSL-10-5600ACTVE®IGF-I ELISA kit and the DSL-10-6600ACTIVE®IGFBP-3 ELISA kit, respectively (DSL, Webster, Tex.), were used. Every sample was run in duplicate and the mean calculated for data analysis. Differences between the two measurements were minimal, as shown by the intra-assay precision coefficient of variation of only 4.59±2.43% for IGF-1 and 6.49±4.42% for IGFBP-3.

Pathological Examination

All histologic slides were reviewed without knowledge of clinical outcome. The 1997 TNM classification was used for clinical and pathologic staging. The WHO classification was used for clinical and pathologic grading. The level of capsular invasion (PCI), with respect to the stroma of the prostate, prostatic capsule, and periprostatic soft tissue, was classified as described previously.

Post-Operative Treatment

Five patients (10%) received adjuvant chemotherapy with MVAC (methotrexate, vinblastine, doxorubicin, cisplatin) after surgery for adverse pathologic characteristics including regional and distant lymph node metastases, or extravesical involvement. Eleven patients (22%) received MVAC for disease progression. For the patients with concomitant prostate cancer, biochemical progression was defined as a sustained elevation, on 2 or more occasions, of PSA>0.2 ng/mL.

Statistical Analysis

Differences in IGF-I and IGFBP-3 levels between clinical and pathological features were tested by the independent sample t-test. Spearman's rank correlation coefficient was used to compare ordinal and continuous variables. Logistic regression was used for multivariate analysis of binary outcome variables. The Kaplan-Meier method was used to calculate survival functions, and differences were assessed with the log rank statistic. Multivariate survival analysis was performed with the Cox proportional hazard regression model. Clinical and pathological stage were evaluated as TIS, Ta, and T1 versus≧T2; clinical and pathologic tumor grade were evaluated as grade 1 and 2 versus grade 3. Statistical significance in this study was set as P<0.05. All reported P values are two-sided. All analyses were performed with SPSS statistical package (SPSS version 10.0 for Windows).

Results

Clinical and Pathological Characteristics as a Function of IGF-I and IGFBP-3

There was no significant difference in mean IGF-I or IGFBP-3 levels between healthy men and bladder cancer patients (P=0.494 and P=0.339). IGF-I and IGFBP-3 levels were strongly associated with each other in bladder cancer patients and in healthy subjects (r=0.568 and r=0.607, respectively, P values≦0.001). Pretreatment IGF-I and IGFBP-3 levels did not correlate with age at time of cystectomy (r=−0.127, P=0.374 and r=−0.108, P=0.450, respectively). The correlation of pre-operative plasma IGF-I and IGFBP-3 levels with various clinical and pathological parameters is described in Table 5. Pre-operative plasma IGFBP-3 levels were significantly lower in patients with regional lymph node involvement (P=0.047). There were no other significant differences in IGF-I or IGFBP-3 levels between clinico-pathologic parameters. In univariate logistic regression analysis, neither pre-operative plasma IGF-I levels nor IGFBP-3 levels were predictors of lymphovascular invasion or advanced pathologic stage (P values≧0.066). In both univariate and multivariate logistic regression analyses that included pre-operative IGF-I, IGFBP-3, clinical stage, and clinical grade, preoperative plasma IGFBP-3 was the sole independent predictor of lymph node involvement (P=0.047; Hazard ratio 0.874, 95% CI 0.765-0.998).

Eighteen (35%) patients had incidental prostate cancer at the time of cystectomy. All except one patient had capsular-confined tumors, and all surgical margins were negative. The Gleason score was 7 in 2 patients and <6 in 16 patients. Post-operative follow-up with serum PSA levels was available in 12 of the patients with concomitant prostate cancer. With a median time follow-up of 13.3 months (range, 6 to 23), none of these patients had experienced biochemical progression.

Bladder Cancer Progression and Survival as a Function of IGF-I and IGFBP-3 and Other Parameters

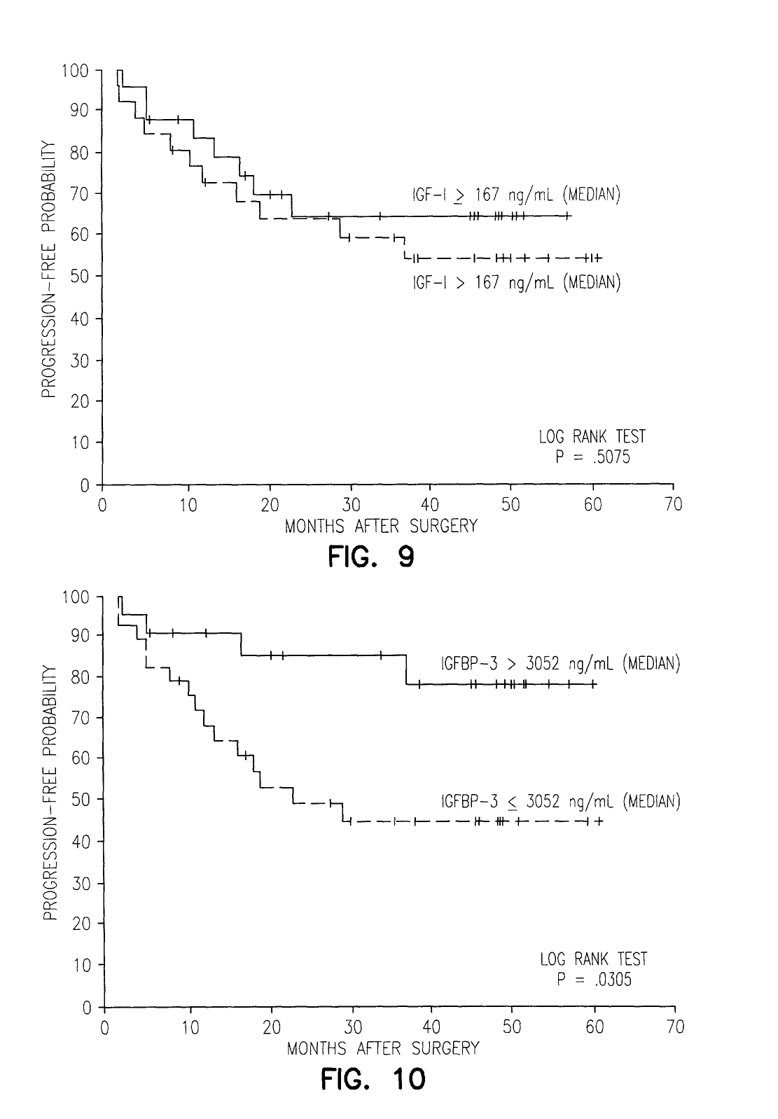

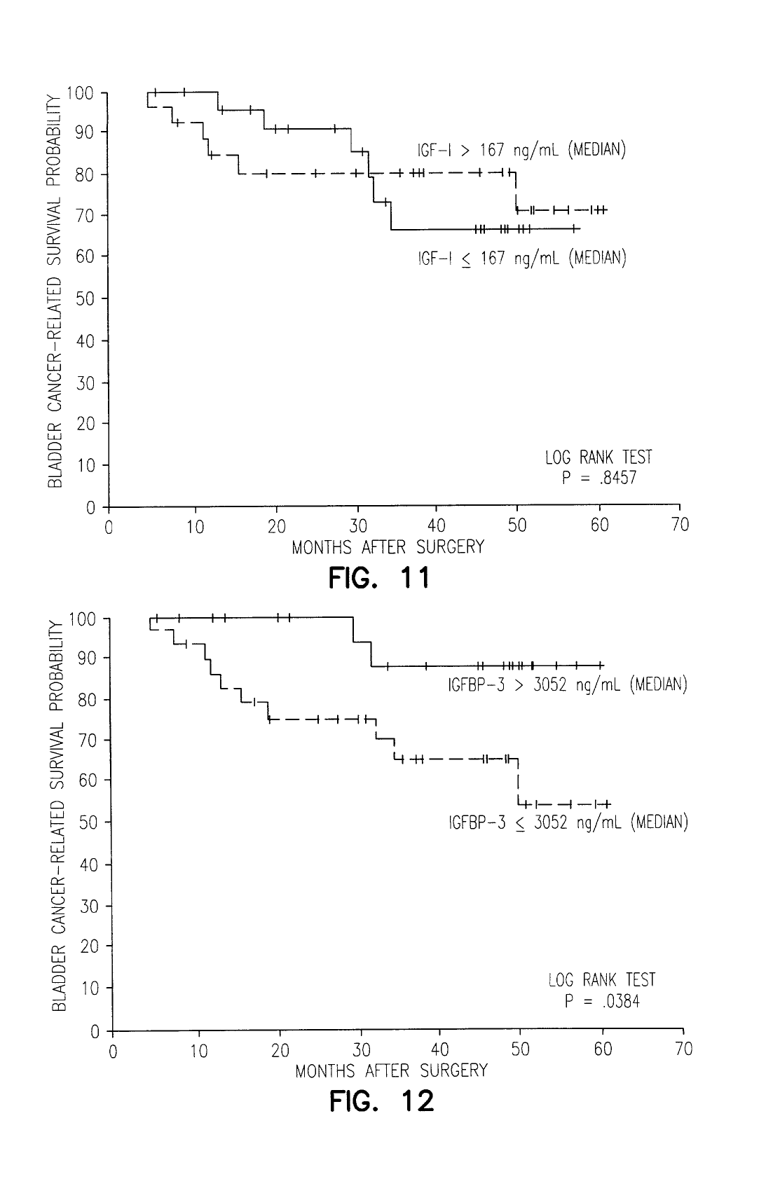

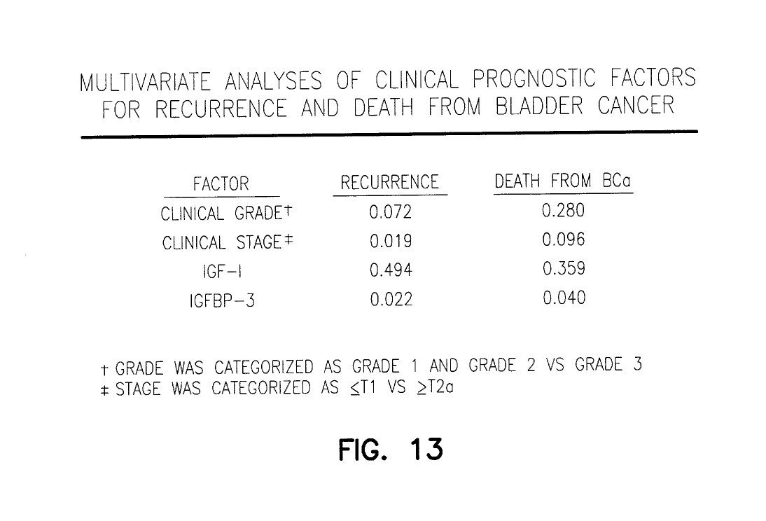

Of 51 patients, 19 (37%) progressed with metastatic disease after cystectomy, and 19 (37%) were dead at the time of analysis. While 12 (63%) of 19 patients died from metastatic bladder cancer, 7 (37%) died of other causes without evidence of disease progression at the time of death. The overall progression-free survival probability was 64.5±7.0% (SE) at 2 years and 59.2±7.3% (SE) at 4 years; the overall disease-specific survival was 85.2±5.2% (SE) at 2 years and 74.3±6.8% (SE) at 4 years. Patients with plasma IGFBP-3 levels below the median (3052 ng/mL) had a significantly increased probability of disease progression (P=0.0305; FIG. 9) and disease-specific mortality (P=0.0384; FIG. 10) compared to patients with IGFBP-3 levels above the median. However, there was no significant difference in progression-free survival (P=0.5075; FIG. 11) and bladder cancer-specific survival (P=0.8457; FIG. 12) between patients stratified by the median level of IGF-I (167 ng/mL).

On univariate Cox proportional hazards regression analyses, pre-operative plasma IGFBP-3 was associated with the risk of progression (P=0.041, Hazard ratio 0.923, 95% CI 0.858-0.993) and of bladder-cancer-related death (P=0.050, Hazard ratio 0.915, 95% CI 0.832-0.998). There was no association of pre-operative plasma IGF-I level with disease progression (P=0.648, Hazard ratio 0.998, 95% CI 0.987-1.008) or disease-specific mortality (P=0.801, Hazard ratio 0.998, 95% CI 0.985-1.012).

In a pre-operative multivariate Cox proportional hazards regression analysis that included IGF-I or IGFBP-3 along with clinical stage and grade, none of the parameters was associated with disease progression or bladder cancer-specific survival (P≧0.054). When both IGF-I and IGFBP-3 were included in the model, IGFBP-3 was independently associated with bladder cancer progression and survival (P=0.050 and P=0.040, respectively; Table 6). In a post-operative multivariate model that included pretreatment IGF-I and IGFBP-3, pathologic grade, pathologic stage, lymphovascular invasion, and lymph node metastases, metastases to regional lymph nodes was the sole independent predictor of bladder cancer progression and survival (P=0.011 and P=0.020, respectively; Table 6).

Discussion

There was no difference in circulating IGF-I or IGFBP-3 levels between healthy subjects and bladder cancer patients treated with radical cystectomy. Plasma IGF-I level was not associated with clinical or pathologic features of bladder cancer or outcome following radical cystectomy. Plasma IGFBP-3 level was, however, an independent predictor of metastases to regional lymph nodes, disease progression, and bladder cancer related mortality, when adjusted for IGF-I.

The independent association of IGFBP-3 with regional lymph node metastasis and prognosis following cystectomy has potentially important clinical implications. The majority of patients with organ-confined bladder cancer and negative nodes, whose local tumor has been completely removed, are reported to have a favorable cancer-specific survival with 5-year actuarial survival rates between 64% and 86%. However, approximately 70% of patients with lymph node involvement eventually fail local therapy and develop distant metastases that lead to death, despite excellent local control with radical cystectomy. Thus, while the pT category of the primary tumor is a substantial stratification variable in determining who may or may not benefit from radical surgery, the nodal tumor burden is the most significant pathologic prognostic factor in patients with bladder TCC. Markers like IGFBP-3 that are associated with lymph node metastases thus may serve a clinically useful role in prospectively identifying patients at high risk for failure with loco-regional therapy alone. Furthermore, IGFBP-3 may serve as a marker for disease activity.

The finding in the post-operative model that only lymph node involvement was independently associated with bladder cancer progression and mortality may be due to the strong association of IGFBP-3 levels with lymph node metastases. The association between lower pre-operative IGFBP-3 levels and disease progression has been reported in patients with prostate cancer, ovarian cancer, and acute childhood leukemia. These data suggest that IGFBP-3 levels are primarily associated with clinically evident or occult metastases present at the time of surgery, that increase the risk of disease progression and bladder cancer-related death.

Viewed in the context of related observations made for other cancers, these data support the existence of a general relationship between circulating IGFBP-3 levels and metastasis. While the liver is the main endocrine source of IGFBP-3, autocrine/paracrine activity is found in most tissues including bladder cancer. IGFBP-3 transports and stabilizes IGFs in the circulation, modulates the effects of IGF on a variety of cellular functions, and regulates cells by IGF independent mechanisms such as induction of apoptosis through binding to its own putative receptor. In addition, studies have shown that IGFBP-3 is able to mediate the anti-proliferative effects of tumor suppressor protein p53, transforming growth factor β, retinoic acid, vitamin D, anti-estrogens, and tumor necrosis factor α. The actions of IGFBP-3 are regulated by a complex system, including IGFBP-3 proteases such as kallikreins, cathepsins, and matrix metalloproteinases, which releases IGFs from IGFBP-3. An increased IGFBP-3 protease activity has been described in patients with advanced breast cancer and has further been associated with the metastatic tumor burden. In the absence of a clear understanding of the complex intracellular mode of action of IGFBP-3 and the overall mechanism relating IGFBP-3 and metastasis, this association might be due to direct tumor factors or to an early host response to the metastatic cascade.

TABLE 5
Pre-operative Plasma IGF-I and IGFBP-3 Levels in Healthy Controls and
Patients Undergoing Radical Cystectomy for Transitional
Cell Carcinoma of the Urinary Bladder

| | | IGF-I (ng/mL) | | | IGFBP-3 (ng/mL) | | |
|---|---|---|---|---|---|---|---|
| | No. of Pts | Median | Range | P^{*} | Median | Range | P^{*} |
| Healthy | 44 | 171 | 62-346 | | 3344 | 1761-5020 | |
| Controls | | | | | | | |
| Bladder Cancer | 51 | 167 | 85-291 | .494 | 3052 | 1884-5452 | .339 |
| Gender | | | | | | | |
| Male | 47 | 167 | 85-291 | | 3052 | 1884-5452 | |
| Female | 4 | 179 | 97-195 | .467 | 3310 | 2062-4844 | .513 |
| Clinical Stage | | | | | | | |
| Superficial | 15 | 160 | 92-220 | | 3052 | 1955-4383 | |
| TIS | 3 | | | | | | |
| Ta | 1 | | | | | | |
| T1 | 11 | | | | | | |
| Invasive | 36 | 167 | 85-291 | .790 | 3011 | 1884-5452 | .592 |
| T2 | 28 | | | | | | |
| T3 | 5 | | | | | | |
| T4a | 3 | | | | | | |
| Clinical Grade | | | | | | | |
| 2 | 6 | 171 | 116-220 | | 2840 | 2151-3776 | |
| 3 | 45 | 167 | 85-291 | .720 | 3052 | 1884-5452 | .569 |
| Pathologic | | | | | | | |
| Stage | | | | | | | |
| Superficial | 10 | 176 | 92-220 | | 3052 | 2399-4383 | |
| PTIS | 2 | | | | | | |
| PT1 | 8 | | | | | | |
| Invasive | 41 | 166 | 85-291 | .789 | 3034 | 1884-5452 | .876 |
| PT2 | 18 | | | | | | |
| PT3 | 14 | | | | | | |
| PT4 | 9 | | | | | | |
| Pathologic | | | | | | | |
| Grade | | | | | | | |
| 2 | 7 | 160 | 98-220 | | 3052 | 1884-3776 | |
| 3 | 44 | 170 | 85-291 | .661 | 3047 | 1955-5452 | .466 |
| Carcinoma insitu | | | | | | | |
| Negative | 25 | 164 | 98-291 | | 3056 | 1884-5452 | |
| Positive | 26 | 167 | 85-274 | .941 | 3034 | 1955-4383 | .314 |
| Lymphovascular | | | | | | | |
| Invasion | | | | | | | |
| Negative | 32 | 174 | 92-291 | | 3133 | 1884-5452 | |
| Positive | 19 | 161 | 85-240 | .283 | 2911 | 1981-4346 | .301 |
| Regional | | | | | | | |
| Lymph Node | | | | | | | |
| Metastases | | | | | | | |
| Negative | 37 | 168 | 92-291 | | 3193 | 1884-5452 | |
| Positive | 14 | 166 | 85-204 | .495 | 2853 | 1981-3842 | .047 |
| Distant Lymph | | | | | | | |
| Node | | | | | | | |
| Metastases† | | | | | | | |
| Negative | 47 | 168 | 85-291 | | 3052 | 1884-5452 | |
| Positive | 4 | 133 | 109-204 | .430 | 2986 | 1981-3842 | .605 |
| Prostate cancer | | | | | | | |
| Negative | 33 | 163 | 85-220 | | 2986 | 1955-4383 | |
| Positive | 18 | 168 | 98-291 | .606 | 3183 | 1884-5452 | .438 |

^{*}Independent sample t-test.
†Above the common iliac bifurcation.

TABLE 6
Multivariate Cox Regression Analysis of Pre-operative and Post-Operative
Features for the Prediction of Disease Progression and Disease-specific Survival of
51 Patients Treated with Radical Cystectomy for Transitional Cell Carcinoma of the
Urinary Bladder
Pre-operative Features

| | Progression | | | Disease-specific survival | | |
|---|---|---|---|---|---|---|
| | Hazard | | | Hazard | | |
| | Ratio | P | 95% CI | Ratio | P | 95% CI |
| Clinical grade^{*} | 4.037 | .072 | 0.880-18.507 | 2.716 | .280 | 0.443-16.657 |
| Clinical stage† | 5.884 | .062 | 0.994-25.874 | 4.482 | .096 | 0.768-26.164 |
| Preoperative IGF-I | 1.004 | .494 | 0.992-1.017 | 1.008 | .359 | 0.991-1.027 |
| levels | | | | | | |
| Preoperative IGFBP-3 | 0.905 | .050 | 0.831-0.999 | .884 | .040 | 0.787-0.994 |
| levels | | | | | | |

Postoperative Features

| | Progression | | | | Disease-specific | |
|---|---|---|---|---|---|---|
| | Hazard | | | | survival | |
| | ratio | P | 95% CI | | P | 95% CI |
| Pathologic grade^{*} | 1.348 | .202 | .269-3.763 | 1.566 | .695 | 0.330-9.754 |
| Pathologic stage† | 1.836 | .646 | .246-9.593 | 1.681 | .795 | 0.370-12.357 |
| Lymphovascular | 2.610 | .100 | 0.832-8.190 | 3.310 | .093 | 0.821-13.346 |
| invasion | | | | | | |
| Lymphnode | 4.198 | .011 | 1.386-12.710 | 8.026 | .020 | 1.388-46.404 |
| metastases | | | | | | |
| Preoperative IGF- | 1.007 | .319 | 0.994-1.019 | 1.004 | .623 | 0.987-1.022 |
| I levels | | | | | | |
| Preoperative | 0.930 | .136 | 0.845-1.023 | 0.947 | .412 | 0.831-1.079 |
| IGFBP-3 levels | | | | | | |

^{*}Clinical and pathological tumor grades were analyzed as dichotomous variables (grade 2 versus grade 3).
†Clinical and pathological tumor stages were analyzed as dichotomous vatiables (≦T1 versus ≧T2a).

### EXAMPLE 4

### Association of Plasma Urokinase-Type Plasminogen Activator and its Receptor with Clinical Outcome in Patients Undergoing Radical Cystectomy for Transitional Cell Carcinoma of the Bladder

Materials and Methods

Patient Population

Pre-operative plasma levels of uPA in 51 patients who were treated with radical cystectomy and pelvic lymphadenectomy for biopsy-proven muscle-invasive or refractory superficial TCC were measured. Plasma uPAR levels in 38 of the 51 patients, for whom plasma samples were available, were also assessed. There were 47 (92%) males and 4 (8%) females, and the mean age of the patients was 65.0±8.5 years (median 67, range 41 to 76) at the time of the cystectomy. The median follow up was 45.7 months (range 4 to 61) for those patients alive at the time of analysis.

Two patients had clinical evidence of secondary cancer prior to cystectomy. One patient had undergone a colon resection for colon cancer 10 years before the cystectomy, and radiation therapy for prostate cancer 13 years prior to cystectomy. The second patient had received radiation therapy for prostate cancer 16 years before the cystectomy. Both patients were free of biochemical and clinical disease progression for prostate cancer and colon cancer prior to cystectomy and at the time of the last follow-up. No other patient was treated either pre-operatively or post-operatively with radiation therapy. One patient was treated with neoadjuvant chemotherapy prior to surgery for metastases to regional lymph nodes detected on a previous lymphadenectomy. Twenty-four patients had been treated previously with intravesical therapy (IVT) including Bacillus Calmette Guerin (BCG), Mitomycin C, and/or Thiotepa. The indications for cystectomy in patients with initial Ta, T1, or Tis bladder TCC included IVT failure (n=6), multiple recurrences (n=5), aggressive histopathologic features (high-grade tumors, presence of CIS, multifocal extensive disease) (n=4), or progression to muscle invasive cancer (n=19). Seventeen patients underwent cystectomy for an initial presentation of muscle invasive disease.

Plasma uPA and uPAR levels were also assessed in 44 healthy patients without cancer. This group was composed of three sets of consecutive patients who participated in the Baylor Prostate Center's weekly prostate cancer screening program. They had no prior history of cancer or chronic disease, a normal digital rectal examination, and a PSA of less than 2.0 ng/mL, a PSA range that has an estimated probability of prostate cancer detection of less than 1% in the first 4 years after screening (Smith et al., 1996).

Pathological Examination

All histologic slides were reviewed without knowledge of clinical outcome. The 1997 TNM classification was used for pathologic staging and the WHO classification was used for pathologic grading. Pathological stages of the prostate cancer found incidentally at the time of cystectomy in 18 (35%) patients were T1 (n=7, 39%), T2 (n=10, 56%), and T3a (n=1, 6%). Pathological Gleason sums were three (n=2, 11%), five (n=6, 33%), six (n=8, 44%), and seven (n=2, 11%) with a median Gleason score of six. uPA and uPAR Measurements Plasma samples were collected on the morning of the scheduled day of surgery after a preoperative overnight fast. Samples were collected from healthy controls at the time of prostate cancer screening. Blood was collected into VACUTAINER CPT 8 mL tubes containing 0.1 mL of 1 M sodium citrate anticoagulant and centrifuged at room temperature for 20 minutes at 1500× g. The top layer was decanted using sterile transfer pipettes and immediately frozen and stored at −80° C. in polypropylene cryopreservation vials (NalgeNunc, Rochester, N.Y.). For quantitative measurements of plasma uPA and uPAR levels, quantitative sandwich enzyme immunoassays (IMUBIND uPA ELISA kit, American Diagnostica, Greenwich, Conn. and QUANTIKINE uPAR Immunoassay, R&D Systems, Minneapolis, Minn.) were used. Samples were measured in duplicate, the mean was calculated and differences between the two measurements for both uPA and uPAR were minimal, as shown by the intra-assay precision coefficients of variation of only 6.3±2.7% and 7.9±4.3%, respectively.

Post-Operative Follow-Up

Five patients (10%) received adjuvant chemotherapy (MVAC) after surgery for adverse pathologic characteristics including regional or distant lymph node metastases, or extravesical involvement. Eleven patients (22%) received MVAC for disease progression. For the patients with concomitant prostate cancer, biochemical progression was defined as a sustained elevation, on 2 or more occasions, of serum PSA>0.2 ng/mL.

Statistical Analysis

Differences in plasma uPA and uPAR levels were assessed using the Mann-Whitney U test. Spearnan's rank correlation coefficient was used to compare age and levels of uPA and uPAR. Logistic regression was used for analyses of binary outcome. Multivariable survival analysis was performed with the Cox proportional hazards regression model. Clinical and pathological stage were evaluated as Tis, Ta, and T1 versus≧T2; clinical and pathologic tumor grades were evaluated as grade 2 versus grade 3. Statistical significance in this study was set as P<0.05. All reported P values are two-sided. All analyses were performed with SPSS statistical package (SPSS version 10.0 for Windows).

Results

Association of Plasma uPA and uPAR with the Diagnosis of Bladder Cancers and its Clinical and Pathologic Characteristics

Table 7 shows the association of plasma uPA and uPAR levels with clinical and pathologic characteristics. Plasma uPA and uPAR levels in bladder cancer patients were both significantly higher than those in healthy controls (P<0.001). In addition, uPA, but not uPAR, levels were significantly elevated in patients with lymphovascular invasion (P=0.019 and P=0.091, respectively) and metastases to regional lymph nodes (P=0.017 and P=0.058, respectively). In contrast, uPAR, but not uPA, levels were significantly elevated in patients with metastases to distant lymph nodes (P=0.042 and P=0.197, respectively). Plasma uPA and uPAR levels were weakly correlated with each other (r=0.304; P=0.042).

In univariable logistic regression analyses, pre-operative uPA was associated with lymphovascular invasion (P=0.016; Hazard ratio 6.231, 95% CI 1.897-18.178) and regional lymph node metastases (P=0.008; Hazard ratio 5.518, 95% CI 2.909-10.466), whereas preoperative uPAR was not (P=0.091 and P=0.059, respectively). In multivariable logistic regression analyses that included pre-operative uPA, clinical stage, and clinical grade, pre-operative plasma uPA was an independent predictor of lymphovascular invasion (P=0.015; Hazard ratio 5.006, 95% CI 1.409-18.262) and metastases to regional lymph node (P=0.017; Hazard ratio 3.862, 95% CI 1.794-10.646).

Association of Pre-Operative Plasma uPA and uPAR with Clinical Outcome

Of 51 patients, 19 patients (37%) had progression of disease, and 19 patients (37%) were dead at the time of analysis. While 12 of 19 patients (63%) died from metastatic bladder cancer, 7 (37%) died of other causes without evidence of disease progression at the time of death. The overall progression-free survival was 59.2±7.3% (SE) at 3 years and at 5 years; the overall disease-specific survival was 74.3±6.8% (SE) at 3 years and 68.4±8.5% (SE) at 5 years.

In univariable Cox proportional hazards regression analyses, pre-operative uPA, but not pre-operative uPAR, was associated with disease progression (P=0.031, Hazard ratio 3.933, 95% CI 1.204-12.272 and P=0.228, Hazard ratio 2.094, 95% CI 0.630-6.962, respectively) and bladder cancer-related mortality (P=0.017, Hazard ratio 4.087, 95% CI 1.330-12.206 and P=0.064, and Hazard ratio 3.355, 95% CI 0.934-12.056, respectively). In a pre-operative multivariable model that included clinical stage, clinical grade, and uPA, pre-operative uPA was independently associated with disease progression (P=0.030) and disease-specific death (P=0.038; Table 8).

Discussion

As described above, plasma uPA and its receptor are significantly elevated in patients with bladder cancer compared to healthy controls. For patients undergoing radical cystectomy, the pre-operative plasma level of uPA was independently associated with lymphovascular involvement, metastases to regional lymph nodes, disease progression, and death from bladder cancer. Pre-operative plasma levels of uPAR were significantly elevated in patients with metastases to distant lymph nodes, but were not associated with any other pathologic feature or clinical outcomes of bladder cancer.

Various sources of production of uPA can contribute to the level found circulating in the plasma. uPA is produced by normal tissues for maintenance and growth purposes, but can also be produced by cancerous tissues (Wang, 2001; Andreason et al., 2000). However, a cause and effect relationship between increased circulating uPA and cancer metastases has not been clearly established. Increased local production of uPA at the primary tumor site may effect local invasion and subsequent development of metastases. Hasui et al. (1996) reported that higher tissue expression of uPA is independently associated with an increased risk of death in patients with Ta and T1 TCC. While no direct correlation between tumor content and blood levels of uPA has been reported, as described herein, plasma uPA levels were significantly higher in patients with bladder cancer than those in healthy subjects. In patients with bladder cancer, uPA levels were higher in patients with features of both local tumor invasion (e.g., lymphovascular invasion) and metastases than in patients without these characteristics. There is ample evidence that higher circulating levels of uPA found in cancer patients are derived predominantly from tumor cells at either the primary site or from metastatic deposits. Blood levels of uPA correlate with tumor volume, and blood levels of uPA decrease after tumor resection. Furthermore, the number of metastases decrease with administration of inhibitors of uPA, and metastasis is enhanced with administration of uPA or transfection of non-metastatic cells with cDNA for uPA (Wang, 2001; Andreasen et al., 2000).

uPA activity is mediated via binding to its specific receptor, uPAR, located on the surface of cells, including bladder cancer cells (Hudson et al., 1997). In addition to its receptor function, upon binding to uPA, uPAR is a pleiotropic ligand for other surface molecules uPA (Wang, 2001; Andreasen et al., 2000; Blassi, 1999). Recent data have suggested that uPAR can by itself mediate chemotaxis of human monocytes and cause profound changes in cytoskeletal organization indicating that uPAR has the properties of a cell-surface regulated chemokine (Blassi, 1999). In bladder cancer cells and cells from other malignancies, the presence of both uPA and uPAR were found to be necessary for in vitro invasion and invasive potential was reduced when the uPA-uPAR interaction was disrupted (Hudson et al., 1997; Reiter et al., 1997; Park et al., 1997). Nevertheless, plasma levels of uPAR were elevated in bladder cancer patients compared to healthy controls, but no association of uPAR levels with pathological characteristics or clinical outcome was found except for with distant lymph node metastases. While studies in different types of cancer found that elevated level of uPAR measured in the primary tumor is associated with poor prognosis, the significance of blood levels of uPAR is not clear. As with uPA, the association between local tumor content and circulating levels of uPAR remains unknown. Neither the source of soluble uPAR in human body fluids nor the mechanism of receptor release from the cell surface has been clearly determined. While blood levels of uPAR were not associated with bladder cancer outcome, the nonsignificant results for uPAR may be due to the small patient population for whom plasma samples were available compared to those who had uPA assayed. Conclusions Pre-operative plasma uPA, but not its receptor, is an independent predictor of lymphovascular invasion, metastases to regional lymph nodes, disease progression and death from bladder cancer after cystectomy. These findings suggest that plasma uPA may be a marker of biologically and clinically aggressive bladder cancers and may help predict behavior following definitive local therapy with radical cystectomy.

TABLE 7
Pre-operative Plasma uPA and uPA Receptor Levels in Patients
Undergoing Radical Cystectomy for Transitional Cell Carcinoma
of the Bladder

| UPA | | | | uPAR | | | |
|---|---|---|---|---|---|---|---|
| No. | Median | Range | | No. | Median | Range | |
| Pts | (ng/mL) | (ng/mL) | P* | Pts | (ng/mL) | (ng/mL) | P* |

| | | | | | | | | |
|---|---|---|---|---|---|---|---|---|
| Healthy Controls | 44 | 0.20 | 0.10-0.30 | | 38 | 1.47 | 1.02-2.51 | |
| Bladder Cancer | 51 | 0.32 | 0.06-0.99 | <0.001 | 38 | 2.09 | 1.18-3.98 | <0.001 |
| Clinical Stage | | | | | | | | |
| Ta, Tis, or T1 | 15 | 0.28 | 0.11-0.82 | 0.926 | 11 | 2.24 | 1.32-2.76 | 0.475 |
| ≧T2a | 36 | 0.32 | 0.06-0.99 | | 27 | 2.07 | 1.18-3.98 | |
| Clinical Grade | | | | | | | | |
| 2 | 6 | 0.54 | 0.11-0.80 | 0.243 | 4 | 2.18 | 2.07-2.69 | 0.482 |
| 3 | 45 | 0.32 | 0.06-0.99 | | 34 | 2.07 | 1.18-3.98 | |
| Pathologic Stage | | | | | | | | |
| Ta, Tis, or T1 | 10 | 0.27 | 0.11-0.82 | 0.803 | 8 | 2.33 | 1.32-2.76 | 0.086 |
| ≧T2a | 41 | 0.32 | 0.06-0.99 | | 30 | 2.07 | 1.18-3.98 | |
| Pathologic Grade | | | | | | | | |
| 2 | 7 | 0.28 | 0.11-0.64 | 0.883 | 5 | 2.24 | 1.64-2.76 | 0.074 |
| 3 | 44 | 0.32 | 0.06-0.99 | | 33 | 2.08 | 1.18-3.98 | |
| Carcinoma in situ | | | | | | | | |
| Negative | 24 | 0.32 | 0.11-0.99 | 0.850 | 17 | 2.20 | 1.40-3.98 | 0.170 |
| Positive | 27 | 0.32 | 0.06-0.88 | | 21 | 2.03 | 1.18-2.76 | |
| Lymphovascular | | | | | | | | |
| Invasion | | | | | | | | |
| Negative | 32 | 0.27 | 0.06-0.82 | 0.019 | 23 | 2.08 | 1.18-2.76 | 0.091 |
| Positive | 19 | 0.44 | 0.10-0.99 | | 15 | 2.12 | 1.42-3.98 | |
| Regional Lymph | | | | | | | | |
| Node Metastases | | | | | | | | |
| Negative | 37 | 0.28 | 0.06-0.82 | 0.017 | 27 | 2.07 | 1.32-2.76 | 0.058 |
| Positive | 14 | 0.52 | 0.10-0.99 | | 11 | 2.24 | 1.18-3.98 | |
| Distant Lymph | | | | | | | | |
| Node Metastases† | | | | | | | | |
| Negative | 47 | 0.32 | 0.06-0.88 | 0.197 | 35 | 2.07 | 1.18-2.76 | 0.042 |
| Positive | 4 | 0.63 | 0.18-0.99 | | 3 | 2.96 | 2.34-3.98 | |
| Sex | | | | | | | | |
| Male | 47 | 0.32 | 0.06-0.99 | 0.853 | 35 | 2.11 | 1.18-3.98 | 0.990 |
| Female | 4 | 0.29 | 0.20-0.66 | | 3 | 1.90 | 1.82-2.60 | |
| Prostate Cancer | | | | | | | | |
| Negative | 32 | 0.36 | 0.10-0.99 | 0.262 | 25 | 2.23 | 1.32-3.98 | 0.053 |
| Positive | 19 | 0.28 | 0.60-0.80 | | 13 | 1.83 | 1.18-2.39 | |

*Mann-Whitney U test
†Above the common iliac bifurcation.

TABLE 8
Multivariable Cox Regression Analysis of Pre-operative
Features for the Prediction of Disease Progression and
Disease-specific Survival of 51 Patients Treated with
Radical Cystectomy for Transitional Cell
Carcinoma of the Bladder

| | Pre-operative Features | | | | | |
|---|---|---|---|---|---|---|
| | Recurrence | | | Disease-specific survival | | |
| | Hazard ratio | 95% CI | P | Hazard ratio | 95% CI | P |
| Clinical grade* | 1.517 | 0.043-5.095 | .104 | 1.285 | 0.041-5.980 | .204 |
| Clinical stage† | 2.292 | 0.916-11.732 | .085 | 2.291 | 0.634-6.070 | .136 |
| Preoperative uPA | 3.726 | 1.128-12.013 | .030 | 3.662 | 1.160-11.338 | .038 |
| levels | | | | | | |

*Clinical tumor grade was analyzed as a dichotomous variable (grade 2 versus grade 3)
†Clinical tumor stage was analyzed as a dichotomous variable (≦T1 versus ≧T2a).

### EXAMPLE 5

### Urinary Level of Urokinase-Type Plasminogen Activator and its Receptor in the Detection of Bladder Carcinoma

Materials and Methods

Patient Population

Two hundred twenty nine (229) patients undergoing cystoscopy were prospectively evaluated as reported in Casella et al. (2000). Informed consent was obtained from each patient. Clinical and pathologic features of the patents are shown in Table 9. Patients were examined because of suspected bladder malignancy, as follow-up after transurethral resection of bladder TCC, or because of other pathological conditions. Ten were healthy volunteers. A voided urine sample was collected prior to cystoscopy in all patients and used for uPA and uPAR measurements. In a subset of 191 subjects (93 with and 98 without bladder TCC), bladder washout samples for cytology were collected during cystoscopy. There were 153 (67%) males and 76 (33%) females, and the mean age was 68.9±13.0 years (median 71, range 21 to 94) at time of specimen collection. One hundred twenty six patients (55%) underwent surgery after cystoscopy (125 transurethral resection of the tumor and one cystectomy). Overall, 122 patients (53%) were found to have a bladder tumor. The 107 patients without a bladder tumor belonged to three different categories: patients with past history of bladder cancer but without tumor evidence at actual cystoscopy (n=66), patients with urological pathology other than bladder malignancy (n=31), and healthy volunteers (n=10).

TABLE 9A
Urinary Levels of uPA and uPAR in 107 Control Subjects and 122
Patients with Transitional Cell Carcinoma of the Urinary Bladder.

| | No. Pts | uPA (ng/mL) | | | uPAR (ng/mL) | | |
|---|---|---|---|---|---|---|---|
| | (%) | Median | Range | P* | Median | Range | P* |
| Control Subjects | 107 (47) | 0.96 | 0.0-33.0 | | 1.19 | 0.0-15.2 | |
| Bladder cancer | 122 (53) | 3.30 | 0.0-34.1 | <0.001 | 1.38 | 0.0-9.0 | 0.016 |
| patients | | | | | | | |
| Pathologic Stage | | | | | | | |
| Ta or Tis | 72 (59) | 2.15 | 0.0-25.2 | | 1.29 | 0.0-7.9 | |
| T1 or higher | 50 (41) | 4.88 | 0.0-34.1 | 0.019 | 1.53 | 0.2-9.0 | 0.235 |
| stage | | | | | | | |
| Pathologic | | | | | | | |
| Grade | | | | | | | |
| 1 or 2 | 85 (70) | 2.24 | 0.0-34.1 | | 1.39 | 0.1-9.0 | |
| 3 | 37 (30) | 4.58 | 0.0-33.0 | 0.048 | 1.25 | 0.0-5.3 | 0.936 |
| Cytology | | | | | | | |
| Negative | 131 (69) | 1.54 | 0.0-33.0 | | 1.21 | 0.0-15.2 | |
| Positive | 60 (31) | 3.40 | 0.0-21.7 | 0.003 | 1.34 | 0.0-9.0 | 0.285 |
| Gender | | | | | | | |
| Male | 153 (67) | 2.14 | 0.0-34.1 | | 1.25 | 0.0-9.1 | |
| Female | 76 (33) | 1.34 | 0.0-33.0 | 0.241 | 1.31 | 0.0-15.2 | 0.902 |

*Mann-Whitney U test

TABLE 9B
Association of Urinary Levels of uPA and uPAR and Selected
Characteristics with Transitional Cell Carcinoma of
the Urinary Bladder

| | No. Pts | Case subjects | Control subjects | |
|---|---|---|---|---|
| | (%) | (n = 122) | (n = 107) | P |
| Age, median | 229 | 73.1 (40.2-94.2) | 69.9 (21.0-86.3) | <.001† |
| (range) | | | | |
| Gender, N (%) | | | | |
| Female | 153 (67) | 34 (28) | 42 (39) | |
| Male | 76 (33) | 88 (72) | 65 (61) | 0.091‡ |
| Cytology, N (%)* | | | | |
| Positive | 60 (31) | 50 (54) | 10 (10) | |
| Negative | 131 (69) | 43 (46) | 88 (90) | <.001‡ |
| uPA, N (%) | | | | |
| Median (range) | 229 (100) | 3.30 (0.0-34.1) | 0.96 (0.0-33.0) | <.001† |
| 1^{st} quartile | 58 (25) | 24 (20) | 34 (32) | |
| 2^{nd} quartile | 56 (25) | 23 (19) | 33 (31) | |
| 3^{rd} quartile | 58 (25) | 32 (26) | 26 (24) | |
| 4^{th} quartile | 57 (25) | 43 (35) | 14 (13) | <.001§ |
| UPAR, N (%) | | | | |
| Median (range) | 229 (100) | 1.38 (0.0-9.0) | 1.19 (0.0-15.2) | 0.016† |
| 1^{st} quartile | 57 (25) | 22 (18) | 35 (33) | |
| 2^{nd} quartile | 57 (25) | 35 (29) | 22 (21) | |
| 3^{rd} quartile | 58 (25) | 29 (24) | 29 (27) | |
| 4^{th} quartile | 57 (25) | 36 (30) | 21 (20) | 0.030§ |

*Urinary cytology was available in 191 patients only.
†Mann-Whitney U test
‡Fisher's exact test
§Chi-Square Test

Pathological Examination and Cytology Grading

All histologic slides were reviewed without knowledge of clinical data. Bladder tumors were staged according to the 1997 TNM classification and assigned a grade according to the WHO classification. Cytological findings were grade 0 (no a typical cells), 1 to 2 (low grade atypia), and 3 (high grade atypia) (Koss, 1992). Only high-grade cytology was considered positive.

uPA and uPAR Measurements

Commercially available quantitative sandwich enzyme immunoassays (IMUBIND 894 and 893, respectively; American Diagnostica, Greenwich, Conn.) were used for quantitative measurement of urinary uPA and uPAR levels. These assays are specific for each of these markers and do not cross-react with structurally similar molecules. Recombinant uPA and uPAR were used as standards. Every sample was run in duplicate and the mean calculated for data analysis. Differences between the two measurements were minimal, as shown by the intra-assay precision coefficient of variation of only 6.54±2.92% for uPA and 7.32±2.83 for uPAR. Statistical Analysis Spearman correlation coefficients were used to examine the correlation between uPA and uPAR levels and among these two protein levels and age. uPA and uPAR levels were analyzed categorically on the basis of their quartile distribution in the case and control subjects combined. The association between categorical data and differences of categorical data between cases and controls were tested using the Fisher's exact test or Chi-square (χ²) test. Differences in age across uPA and uPAR quartile categories were assessed using Kruskal-Wallis nonparametric analysis of variance. Since age had a skewed distribution, differences in age between cases and controls were tested using the Mann-Whitney U test. Univariable and multivariable logistic regression models were used to calculate odds ratio and its 95% confidence interval (CI). The lowest quartile was used as the referent category when calculating the odds ratios. Tests for linear trend were performed using quartile levels as continuous variables. Age had a skewed distribution and therefore was modeled with a logarithmical transformation.

Tumor stage was evaluated as Tis and Ta versus≧T1; tumor grade was evaluated as grade 1 and 2 versus grade 3. Statistical significance in this study was set as P<0.05. All reported P values were two-sided. All analyses were performed with SPSS statistical package (SPSS version 10.0 for Windows).

Results

Association of Urinary Levels of uPA and uPAR and of Urinary Cytology with Clinical and Pathologic Characteristics

Clinical and pathologic characteristics of 229 subjects and the associations of the characteristics with urinary uPA and uPAR levels are shown in Tables 9A and B. Urinary levels of uPA and uPAR were significantly higher in patients with bladder tumors than in healthy controls (P<0.001 and P=0.016, respectively). Urinary levels of uPA were moderately but significantly correlated with uPAR levels (r=0.442, P<0.001). There was no correlation between both urinary uPA and uPAR levels and age at time of specimen collection (r=0.180, P=0.098 and r =0.166, P=0.121). There was no difference in age across uPA and uPAR quartiles (P=0.067 and P=0.078, respectively). Association of clinical and pathologic characteristics including urinary levels of uPA and uPAR with bladder cancer is shown in Table 9. There were significant differences between cases and controls in the quartile distributions for uPA and uPAR levels. For both uPA and uPAR, the distribution in cases was skewed towards the highest quartile compared to controls (P<0.001 and P=0.030). The categorical distribution of urinary uPA and uPAR levels across selected clinical and pathologic characteristics is shown in Table 10. Urinary levels of uPA were higher in patients with abnormal urine cytology (P=0.006). Abnormal urinary cytology was associated with both invasive tumor stage, (in 25 of the 64 Ta TCC or Tis patients versus 25 of the 29≧T1 TCC patients, P<0.001) and with higher tumor grade (in 27 of the 69 Grade 1 or 2 patients versus 23 of the 24 Grade 3 patients, P<0.001). Age was higher in patients with positive cytology than those with negative cytology (P=0.023). However, cytology was not associated with gender (results were positive in 43 of the 132 males and 17 of the 59 females, P=0.736.)

Prediction of Presence of Bladder Cancer and Pathologic Stage and Grade

In univariate logistic regression analyses (Table 10), higher urinary levels of uPA and uPAR, and positive urinary cytology were associated with an increased risk of TCC of the bladder (P for trend<0.001, P for trend=0.34, and P<0.001, respectively. In a multivariable logistic regression model (Table 10), only higher urinary uPA and positive cytology were associated with the presence of bladder cancer (P for trend=0.031 and P<0.001, respectively), when adjusted for the effects of urinary uPAR and age.

TABLE 10A
Categorical Distributions of Urinary Levels of uPA and uPAR
Across Selected Characteristics

| | | Quartile | | | | |
|---|---|---|---|---|---|---|
| | No. Pts (%) | 1 | 2 | 3 | 4 | P‡ |
| UPA | | | | | | |
| Gender, N (%) | | | | | | |
| Female | 153 (67) | 22 (29) | 20 (26) | 17 (22) | 17 (22) | |
| Male | 76 (33) | 36 (24) | 36 (24) | 41 (27) | 40 (26) | 0.698 |
| Cytology, N (%)* | | | | | | |
| Positive | 60 (31) | 8 (13) | 14 (23) | 15 (25) | 23 (38) | |
| Negative | 131 (69) | 40 (31) | 34 (26) | 34 (26) | 23 (18) | 0.006 |
| Pathologic Stage† | | | | | | |
| Ta or Tis | 72 (59) | 18 (25) | 14 (19) | 19 (26) | 21 (29) | |
| T1 or higher stage | 50 (41) | 6 (12) | 9 (18) | 13 (26) | 22 (44) | 0.220 |
| Pathologic Grade† | | | | | | |
| 1 or 2 | 85 (70) | 20 (24) | 17 (20) | 22 (26) | 26 (31) | |
| 3 | 37 (30) | 4 (11) | 6 (16) | 10 (27) | 17 (46) | 0.256 |
| UPAR | | | | | | |
| Gender, N (%) | | | | | | |
| Female | 153 (67) | 22 (29) | 15 (20) | 20 926) | 19 (25) | |
| Male | 76 (33) | 35 (23) | 42 (28) | 38 (25) | 38 (25) | 0.570 |
| Cytology, N (%)* | | | | | | |
| Positive | 60 (31) | 11 (18) | 17 (28) | 18 (30) | 14 (23) | |
| Negative | 131 (69) | 37 (28) | 31 (24) | 31 (24) | 32 (24) | 0.452 |
| Pathologic Stage, N (%)† | | | | | | |
| Ta or Tis | 72 (59) | 15 (21) | 20 (28) | 17 (24) | 20 (28) | |
| T1 or higher stage | 50 (41) | 7 (14) | 15 (30) | 12 (24) | 16 (32) | 0.802 |
| Pathologic Grade, N (%)† | | | | | | |
| 1 or 2 | 85 (70) | 16 (19) | 22 (26) | 21 (25) | 26 (31) | |
| 3 | 37 (30) | 6 (16) | 13 (35) | 8 (22) | 10 (27) | 0.782 |

*Urinary cytology was available in 191 patients only.
†Pathologic stage and grade were available only in the 122 patients who underwent surgery.
‡Chi-Square Test

TABLE 10B
Univariable and Multivariable Logistic Regression
Analysis of Urinary Levels of uPA and uPAR,
Urinary Cytology, and Age for the Prediction of the
Presence of Transitional Cell Carcinoma of the Bladder

| | Univariable | | | Multivariable | | |
|---|---|---|---|---|---|---|
| | Odds ratio | 95% CI | P | Odds ratio | 95% CI | P |
| UPA | | | | | | |
| 1^{st} quartile | 1.000 | Referent | | 1.000 | Referent | |
| 2^{nd} quartile | 0.982 | 0.460-2.094 | 0.962 | 1.265 | 0.486-3.292 | 0.320 |
| 3^{rd} quartile | 2.353 | 0.837-5.672 | 0.137 | 2.159 | 0.988-4.720 | 0.054 |
| 4^{th} quartile | 4.192 | 1.916-9.169 | <.001 | 3.022 | 1.295-7.054 | 0.011 |
| Test for trend | | | <.001 | | | 0.031 |
| UPAR | | | | | | |
| 1^{st} quartile | 1.000 | Referent | | 1.000 | Referent | |
| 2^{nd} quartile | 1.609 | 0.761-3.402 | 0.213 | 1.171 | 0.614-3.016 | 0.346 |
| 3^{rd} quartile | 2.542 | 1.193-5.413 | 0.016 | 2.030 | 0.947-5.232 | 0.065 |
| 4^{th} quartile | 2.733 | 1.280-5.835 | 0.009 | 2.586 | 1.014-6.594 | 0.047 |
| Test for trend | | | 0.034 | | | 0.168 |
| Age* | 1.003 | 0.999-1.007 | 0.112 | 0.990 | 0.979-1.001 | 0.091 |
| Cytology† | 10.231 | 4.734-22.111 | <0.001 | 10.182 | 4.451-23.291 | <0.001 |

*Age had a skewed distribution and therefore was modeled with a logarithmical transformation.
†Urinary cytology was analyzed as dichotomous variable (grade 1 and 2 versus grade 3).

Discussion

Higher urinary uPA levels were associated with the presence of bladder cancer after adjusting for effects of urinary uPAR levels, urinary cytology, and age. In addition, patients with a positive urinary cytology had higher uPA levels. While no association between urinary levels of uPA and clinical stage and grade was found, only 14% of the TCC patients (18/122) had muscle invasive disease and only 30% had grade 3 disease.

Although urinary uPAR levels were higher in patients with bladder TCC than in healthy subjects, urinary uPAR was not an independent predictor of the presence of bladder cancer. Furthermore, no association was found between urinary uPAR levels and any clinical and pathologic characteristics of bladder cancer. Hudson et al. (1997; 1999) demonstrated that uPAR was necessary for in vitro invasion of bladder cancer cells. However, in accordance with the findings in the present study, no association was found in plasma uPAR levels with clinical and pathologic features of biologically aggressive bladder cancer and clinical outcome, besides metastases to distant lymph nodes, in patients undergoing radical cystectomy (Example 4). It is possible that uPAR is important in local progression, but its soluble levels in blood or urine do not seem to be associated with bladder cancer.

The high sensitivity of cytology in this study is due to the fact that all urine samples were evaluated by a single expert cytopathologist. In addition, bladder washout specimens were used, which have been shown to yield a higher sensitivity than voided urine specimens (Murphy et al., 1984). In contrast to the high sensitivity, the specificity of cytology described above was lower than that reported previously (Saad et al., 2001). All ten patients who had a presumed false positive urinary cytology had a past history of bladder cancer. Five of the ten patients underwent multiple random biopsies of the bladder without evidence of cancer and remained tumor-free at follow-up. These patients remain at high risk for TCC and must be aggressively followed, and upper tract tumors and prostate urethra CIS must be ruled out. Recent data from the Southwest Oncology Group trial of maintenance BCG demonstrates that cytology may convert to normal up to 6 months or longer after a single induction course of BCG (Lamm et al., 2000). Two of the five patients that were not biopsied later developed bladder cancer. The first patient developed a Ta grade 3 TCC two years later, and the second patient developed a Ta grade 1 TCC only seven months after inclusion in the study. Interestingly, his urine was positive for cytology, uPA and uPAR, supporting the potential role of these urine markers for predicting the subsequent occurrence of bladder cancer. Possibly, this patient may also represent a “false negative” of cystoscopy. Although considered as the gold standard for diagnosis, cystoscopy has a false negative rate up to 20%, due either to operator error or to small areas of carcinoma in situ, which may be difficult to detect (van der Poel et al., 2001).

Conclusions

Expert cytologic evaluation of bladder wash specimens plays a central role in the evaluation and management of patients at risk for or with a history of bladder cancer. Urinary levels of uPA, but not uPAR, add independent information to cytology in the detection of bladder cancer as urinary levels of uPA are related to the risk of bladder cancer.

### REFERENCES

Adler H L, McCurdy M A, Kattan M W, et al: Elevated levels of circulating interleukin-6 and transforming growth factor-beta in patients with metastatic prostatic carcinoma. J Urol 161:182-187, 1999.

Akimoto, S., Okumura, A., Fuse, H.: Relationship between serum levels of interleukin-6, tumor necrosis factor-alpha and bone turnover markers in prostate cancer patients. Endocr J. 45,183, 1998.

Amling C L, Thrasher J B, Frazier H A, et al: Radical cystectomy for stages Ta, Tis and T1 transitional cell carcinoma of the bladder. J Urol 151:31-36; 1994.

Andreasen P A, Egelund R and Petersen H H: The plasminogen activation system in tumor growth, invasion, and metastasis. Cell Mol Life Sci. 57: 25-40., 2000.

Belluco, C., Nitti, D., Frantz, M., et al: Interleukin-6 blood level is associated with circulating carcinoembryonic antigen and prognosis in patients with colorectal cancer. Ann Surg Oncol, 7, 133, 2000.

Blasi F: The urokinase receptor. A cell surface, regulated chemokine. Apmis. 107: 96-101, 1999.

Buszello H, Muller-Mattheis V, Ackermann R: Value of computerized tomography in detection of lymph node metastases in bladder cancer. Urology A 33:243-6, 1994.

Cardillo, M. R., Sale, P., and Di Silverio, F.: Heat shock protein-90, IL-6 and IL-10 in bladder cancer. Anticancer Res, 20, 4579, 2000.

Casella R, Huber P, Blochlinger A, et al. Urinary level of nuclear matrix protein 22 in the diagnosis of bladder cancer: experience with 130 patients with biopsy confirmed tumor. J Urol., 164:1926-1928, 2000.

Catalona W J, Smith D S: Cancer recurrence and survival rates after anatomic radical retropubic prostatectomy for prostate cancer: intermediate-term results. J Urol 160:2428-2434, 1998.

Chung, T. D., Yu, J. J., Spiotto, M. T. et al: Characterization of the role of IL-6 in the progression of prostate cancer. Prostate, 38:199, 1999.

Danielpour D, et al.: Evidence for differential regulation of TGFB1 and TGFB2 expression in vivo by sandwich enzyme-linked immunosorbent assays. Annals New York Academy of Sciences, pp. 300-302, 1990.

Danielpour D, et al.: Immunodetection and quantitation of the two forms of transforming growth factor-beta (TGF-B1 and TGF-B2) secreted by cells in culture. Journal of Cellular Physiology 138: 79-86, 1989.

Dasch J R, et al.: Capture immunoassays specific for TGF-B1 and TGF-B2: use in pharmacokinetic studies. Annals New York Academy of Sciences, pp. 303-305, 1990.

Eastham J, Scardino P: Radical prostatectomy for clinical stage T1 and T2 prostate cancer. In: N. J. Vogelzang, P. T. Scardino, W. U. Shipley, and D. S. Coffey (eds.), Comprehensive textbook of genitourinary oncology, pp. 722-738. Maryland: Williams and Wilkins, 2000.

Eder I E, Stenzl A, Hobisch A, et al: Transforming growth factors-beta I and beta 2 in serum and urine from patients with bladder carcinoma [see comments]. J Urol 156:953-957, 1996.

Flanders K C: Transforming Growth Factor-B 1: Histochemical localization with antibodies to different epitopes. The Journal of Cell Biology 108: 653-660, 1989.

Frazier H A, Robertson I E, Dodge R K, et al: The value of pathologic factors in predicting cancer-specific survival among patients treated with radical cystectomy for transitional cell carcinoma of the bladder and prostate. Cancer 71:3993-4001, 1993.

Gohji, K., Fujimoto, N., Fujii, A. et al: Prognostic significance of circulating matrix metalloproteinase-2 to tissue inhibitor of metalloproteinases-2 ratio in recurrence of urothelial cancer after complete resection. Cancer Res 56: 3196, 1996.

Gohji, K., Nomi, M., Niitani, Y. et al: Independent prognostic value of serum hepatocyte growth factor in bladder cancer. J Clin Oncol, 18:2963, 2000.

Greenlee R T, Murray T, Bolden S, et al: Cancer statistics, 2000. CA Cancer J Clin 50:7-33, 2000.

Hasui Y, Marutsuka K, Asada Y, Osada Y. Prognostic value of urokinase-type plasminogen activator in patients with superficial bladder cancer. Urology 47:34-37, 1996.

Hautmann R E, Paiss T: Does the option of the ileal neobladder stimulate patient and physician decision toward earlier cystectomy? J Urol 159:1845-1850, 1998.

Hudson M A, McReynolds L M. Urokinase and the urokinase receptor: association with in vitro invasiveness of human bladder cancer cell lines. J Natl Cancer Inst., 89:709-717, 1997.

Hudson M A, McReynold L M. Urokinase (u-PA) and the u-PA receptor. Modulation of in vitro invasiveness of human bladder cancer cell lines. Adv Exp Med Biol., 462:399-412, 1999.

Ivanovic V, Melman A, Davis-Joseph B, et al: Elevated plasma levels of TGF-beta1 in patients with invasive prostate cancer [letter]. Nat Med 1:282-284, 1995.

Jones, S. A., Horiuchi, S., Topley, N. et al: The soluble interleukin 6 receptor: mechanisms of production and implications in disease. FASEB J, 15:43, 2001.

Junker U, Knoefel B, Nuske K, et al: Transforming growth factor beta 1 is significantly elevated in plasma of patients suffering from renal cell carcinoma. Cytokine 8:794-798, 1996.

Kakehi Y, Oka H, Mitsumori K, et al: Elevation of serum transforming growth factor beta 1 level in patients with metastatic prostate cancer. Urol Oncol 2:131-135, 1996.

Kim J H, Shariat S F, Menesses A D, et al: Transforming growth factor B1 overexpression is associated with loss of receptor expression and clinical outcome of bladder cancer. Proceedings of the American Association for Cancer Research, 2001.

Kinoshita, T., Ito, H., Miki, C.: Serum interleukin-6 level reflects the tumor proliferative activity in patients with colorectal carcinoma. Cancer, 85: 2526, 1999.

Kong F, Jirtle R L, Huang D H, et al: Plasma transforming growth factor-beta1 level before radiotherapy correlates with long term outcome of patients with lung carcinoma. Cancer 86:1712-1719, 1999.

Koss L G. Tumors of the urinary tract and prostate in urinary sediment. Diagnostic Cytology and Its Histopathologic Bases, ed. 4, vol. 2 Philadelphia: J B Lippincott Co., 951-963, 1992.

Lamm D L, Blumenstein B A, Crissman J D, et al. Maintenance bacillus Cahnette-Guerin immunotherapy for recurrent TA, T1 and carcinoma in situ transitional cell carcinoma of the bladder: a randomized Southwest Oncology Group Study. J Urol., 163:1124-1129, 2000.

Lerner S P, Skinner D G: Radical cystectomy for bladder cancer, in Vogelzang N J, Shipley W U, Scardino P T, et al (eds): Comprehensive Textbook of Genitourinary Oncology. Philadelphia, Williams and Wilkins, pp. 425-447, 1999.

Lerner S P, Skinner D G, Lieskovsky G, et al: The rationale for en bloc pelvic lymph node dissection for bladder cancer patients with nodal metastases: long-term results. J Urol 149:758-65, 1993.

Lu, C., Kerbel, R. S.: Interleukin-6 undergoes transition from paracrine growth inhibitor to autocrine stimulator during human melanoma progression. J Cell Biol, 120:1281, 1993.

Lucas C, et al.: The autocrine production of transforming growth factor-B1 during lymphocyte activation. The Journal of Immunology 145: 1415-1422, 1990.

Maulard-Durdux, C., Toubert, M. E., Hennequin C. et al: Serum tissue polypeptide antigen in bladder cancer as a tumor marker: a prospective study. J Clin Oncol, 15:3446, 1997.

Motro, B., Itin, A., Sachs, L. et al: Pattern of interleukin 6 gene expression in vivo suggests a role for this cytokine in angiogenesis. Proc Natl Acad Sci, 87: 3092, 1990.

Murphy W M, Soloway M S, Jukkola A F, Crabtree W N, Ford K S. Urinary cytology and bladder cancer. The cellular features of transitional cell neoplasms. Cancer., 53:1555-1565, 1984.

Nakashima, J., Tachibana, M., Horignchi, Y. et al: Serum interleukin 6 as a prognostic factor in patients with prostate cancer. Clin Cancer Res, 6:2702, 2000.

Nurmi M, Katevuo K, Puntala P: Reliability of CT in preoperative evaluation of bladder carcinoma. Scand J Urol Nephrol 22:125-128, 1988.

Okamoto, M., Hattoff, K., Oyasu, R.: Interleukin-6 functions as an autocrine growth factor in human bladder carcinoma cell lines in vitro. Int J Cancer, 72:149, 1997.

Pagano F, Bassi P, Galetti T P, et al: Results of contemporary radical cystectomy for invasive bladder cancer: a clinicopathological study with an emphasis on the inadequacy of the tumor, nodes and metastases classification. J Urol 145:45-50, 1991.

Park I K, Kim B J, Goh Y J, Lyu M A, Park C G, Hwang E S and Kook Y H: Co-expression of urokinase-type plasminogen activator and its receptor in human gastric-cancer cell lines correlates with their invasiveness and tumorigenicity. Int J Cancer 71:867-73, 1997.

Persad R, Kabala J, Gillatt D, et al: Magnetic resonance imaging in the staging of bladder cancer. Br J Urol 71:566-573, 1993.

Raghavan D, Shipley W U, Gamick M B, et al: Biology and management of bladder cancer [see comments]. N Engl J Med 322:1129-1138, 1990.

Reiter L S, Spertini O and Kruithof E K: Plasminogen activators play an essential role in extracellular-matrix invasion by lymphoblastic T cells. Int J Cancer. 70: 461-6, 1997.

Saad A, Hanbury D C, McNicholas T A, Boustead G B, Woodman A C. The early detection and diagnosis of bladder cancer: a critical review of the options. Eur Urol., 39:619-633, 2001.

Scambia, G., Testa, U., Benedetti Panici, P. et al: Prognostic significance of interleukin 6 serum levels in patients with ovarian cancer. Br J Cancer, 71, 354, 1995.

Seguchi, T., Yokokawa, K., Sugan, H. et al: Interleukin-6 activity in urine and serum in patients with bladder carcinoma. J Urol, 148:791, 1992.

Sehgal, P. B.: Interleukin 6 in infection and cancer. Proc Soc Exp Biol Med, 195: 183, 1990.

Shariat, S., Kim, J. H., Andrews, B. et al: Higher pre-operative plasma levels of interleukin-6 and its soluble receptor are strong predictors of disease progression in patients undergoing radical prostatectomy. Urology 2001a.

Shariat, S., Shalev, M., Menesses-Diaz, A. et al: Pre-operative plasma Level of transforming growth factor beta1 strongly predict progression in patients undergoing radical prostatectomy. J Clin Oncol 2001c.

Shirai Y, Kawata S, Tamura S, et al: Plasma transforming growth factor-beta1 in patients with hepatocellular carcinoma. Comparison with chronic liver diseases. Cancer 73:2275-279, 1994.

Smith D S, Catalona W J, Herschman J D: Longitudinal screening for prostate cancer with prostate-specific antigen [see comments]. JAMA 276:1309-1315, 1996.

Takenawa, J., Kaneko, Y., Fukumoto, M. et al: Enhanced expression of interleukin-6 in primary human renal cell carcinomas. J Natl Cancer Inst, 83:1668, 1991.

Tamm, I., Cardinale, I., Krueger, J. et al: Interleukin 6 decreases cell-cell association and increases motility of ductal breast carcinoma cells. J Exp Med, 170:1649, 1989.

Thompson N L, et al.: Expression of transforming growth factor-B1 in specific cells and tissues of adult and neonatal mice. The Journal of Cell Biology 108: 661-669, 1989.

Thrasher J B, Crawford E D: Current management of invasive and metastatic transitional cell carcinoma of the bladder. J Urol 149:957-972, 1993.

Ueda, T., Shimada, E., Urakawa, T.: Serum levels of cytokines in patients with colorectal cancer: possible involvement of interleukin-6 and interleukin-8 in hematogenous metastasis. J Gastroenterol, 29:423, 1994.

van der Poel H G, Debruyne F M. Can biological markers replace cystoscopy? An update. Curr Opin Urol., 11:503-509, 2001.

Voges G E, Tauschke E, Stockle M, et al: Computerized tomography: an unreliable method for accurate staging of bladder tumors in patients who are candidates for radical cystectomy. J Urol 142:972-974, 1989.

von der Maase H, Hahsen S W, Roberts J T, et al: Gemcitabine and cisplatin versus methotrexate, vinblastine, doxorubicin, and cisplatin in advanced or metastatic bladder cancer: results of a large, randomized, multinational, multicenter, phase III study. J Clin Oncol 18:3068-3077, 2000.

Wang Y: The role and regulation of urokinase-type plasminogen activator receptor gene expression in cancer invasion and metastasis. Med Res Rev. 21:146-70, 2001.

Wheeler T M, Dillioglugil O, Kattan M W, et al: Clinical and pathological significance of the level and extent of capsular invasion in clinical stage T1-2 prostate cancer. Hum Pathol 29:856-862, 1998.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to determine the prognosis of a patient after local therapy for bladder cancer, comprising:
a) contacting a blood plasma sample from a patient prior to local therapy for bladder cancer with an agent that specifically binds to TGF-β₁, IGFBP-3, IL-6, IL-6sR, uPA or uPAR so as to form a complex; and
b) determining or detecting the amount or level of complex formation, and correlating the amount or level of complex formation with the risk of metastases to a lymph node, lymphovascular invasion, disease recurrence, or bladder cancer-specific death in the patient.

2. A method to determine the prognosis of a patient after local therapy for bladder cancer, comprising:
a) contacting a urine sample from a patient prior to local therapy for bladder cancer with an agent that specifically binds to uPA so as to form a complex; and
b) determining or detecting the amount or level of complex formation, and correlating the amount or level of complex formation with the risk of metastasis to a lymph node, lymphovascular invasion, disease recurrence or bladder cancer-specific death in the patient.

3. The method of claim 1 or 2 wherein the agent is an antibody.

4. The method of claim 3 wherein the antibody is a polyclonal antibody.

5. The method of claim 3 wherein the antibody is a monoclonal antibody.

6. The method of claim 1 wherein the local therapy is radical cystectomy.

7. The method of claim 1 wherein the patient received chemotherapy or intravesical therapy prior to local therapy.

8. The method of claim 1 wherein the local therapy is radiation therapy.

9. The method of claim 1 further comprising:
c) contacting a second blood sample obtained from the patient at a point in time after local therapy with the agent so as to form a complex; and
d) comparing complex formation in a) to complex formation in c).

10. The method of claim 2 further comprising:
c) contacting a second urine sample obtained from the patient at a point in time after local therapy with the agent so as to form a complex; and
d) comparing complex formation in a) to complex formation in c).

11. The method of claim 1 further comprising determining or detecting the amount or level of molecules other than TGF-β₁, IGFBP-3, IL-6, IL-6sR, uPA or uPAR which are markers for bladder cancer.

12. The method of claim 2 further comprising determining or detecting the amount or level of molecules other than uPA which are markers for bladder cancer.

13. The method of claim 1 wherein the amount or level of TGF-β₁ is determined or detected.

14. The method of claim 1 wherein the amount or level of IGFBP-3 is determined or detected.

15. The method of claim 1 wherein the amount or level of IL-6 is determined or detected.

16. The method of claim 1 wherein the amount or level of IL-6sR is determined or detected.

17. The method of claim 1 or 2 wherein the amount or level of uPA is determined or detected.

18. The method of claim 1 wherein the amount or level of uPAR is determined or detected.

19. The method of claim 11 or 12 wherein the other molecule is a serum protein.

20. The method of claim 1 or 2 wherein complex formation is detected or determined with an agent that specifically binds the complex.

21. The method of claim 1 wherein complex formation is determined or detected with a second agent that binds TGF-pi.

22. The method of claim 1 wherein complex formation is determined or detected with a second agent that binds IGFBP-3.

23. The method of claim 1 wherein complex formation is determined or detected with a second agent that binds IL-6.

24. The method of claim 1 wherein complex formation is determined or detected with a second agent that binds IL-6sR.

25. The method of claim 1 or 2 wherein complex formation is determined or detected with a second agent that binds uPA.

26. The method of claim 1 or 2 wherein complex formation is determined with a second agent that binds uPAR.

27. The method of any one of claims 20 to 26 wherein the second agent is an antibody.

28. The method of claim 1 or 2 wherein the agent is detectably labeled or binds to a detectable label.

29. The method of claim 20 wherein the agent that binds the complex is detectably labeled or binds to a detectable label.

30. The method of claim 27 wherein the antibody is detectably labeled or binds to a detectable label.

31. The method of claim 1 or 2 wherein the correlating is conducted by a computer.

32. An apparatus, comprising:
a data input means, for input of test information comprising the level or amount of at least one protein in a physiological fluid sample obtained from a mammal, wherein the protein is selected from the group consisting of TGF-β₁, IGFBP-3, IL-6, IL-6sR, uPA and uPAR;
a processor, executing a software for analysis of the level or amount of the at least one protein in the sample;
wherein the software analyzes the level or amount of the at least one protein in the sample and provides the risk of metastasis to a lymph node, lymphovascular invasion, disease recurrence or bladder cancer-specific death in the mammal.

33. The apparatus of claim 32 wherein the amount or level is input manually using the data input means.

34. The apparatus of claim 32 wherein the software constructs a database of the test information.

35. A method to determine the prognosis of a mammal after local therapy for bladder cancer, comprising:
a) inputting test information to a data input means, wherein the information comprises the level or amount of at least one protein in a physiological fluid sample obtained from a mammal, and wherein the protein is selected from the group consisting of TGF-β₁, IGFBP-3, IL-6, IL-6sR, uPA and uPAR;
b) executing a software for analysis of the test information; and
c) analyzing the test information so as to provide the risk of metastasis to a lymph node, lymphovascular invasion, disease recurrence or bladder cancer-specific death in the mammal.

36. A method to diagnose bladder cancer in a patient, comprising:
a) contacting a blood plasma sample from a patient with an agent that binds to IL-6 so as to form a complex; and
b) correlating the amount or level of complex formation with the presence or absence of bladder cancer.

37. A method to diagnose bladder cancer in a patient, comprising:
a) contacting a urine sample from a patient with an agent that binds to uPA so as to form a complex; and
b) correlating the amount or level of complex formation with the presence or absence of bladder cancer.
